(19) United States
(12) Patent Application Publication
(10) Pub. No.: US 2010/0143274 A1
(43) Pub. Date: Jun. 10, 2010

(54) USE OF AMINES AND AMIDES FOR THE STABILIZATION OF ORGANIC MICRONIZED UV ABSORBERS

(75) Inventors: Cyrille Deshayes, Rosenau (FR); Thomas Ehlis, Freiburg (DE); Julie Grumelard, Huningue (FR)

Correspondence Address: BASF Performance Products LLC;Patent Department, 540 White Plains Road, P.O. Box 2005, Tarrytown, NY, 10591, US

(73) Assignee: CIBA CORPORATION, TARRYTOWN, NY (US)

(21) Appl. No.: 12/529,573

(22) PCT Filed: Feb. 27, 2008
(86) PCT No.: PCT/EP2008/052336

§ 371 Date: Jan. 21, 2010

(30) Foreign Application Priority Data
Mar. 7, 2007 (EP) 07103670.1

(51) Int. Cl. A61K 8/35; A61Q 17/04

(52) U.S. Cl. 424/59

(57) ABSTRACT

Disclosed is the use of amines and amides for the stabilization of a cosmetic composition comprising: (a) an organic micronized UV absorber selected from a benzophenone derivative and (b) a dibenzoylmethane derivative. The use of the amines and amides results in the stabilization of the end-product formulations.

The present invention relates to a method of stabilization of organic micronized UV absorbers, specific cosmetic compositions and the use of these cosmetic composition for preventing the human hair or skin from the harmful effect of UV radiation

Cosmetic compositions comprising micronized amino substituted hydroxyphenyl benzophenone derivatives (as described for example in WO 2004/052837) and Butyl methoxydibenzoylmethane (Avobenzone) represent very effectual UV absorber compositions.

However, mixing together these specific UV actives results in the formation of new and larger scaled crystals and/or particles.

This crystallization affects the efficacy of the UV-filter combination and the sensory aspect of the end-product

Surprisingly it has been found that the crystallization can be slowed, limited and/or prevented by the use of amines, amides, alkanolamides and more preferably alkanolamines in the micronized product or within the end-product formulation. As a result, a stabilization of the end-product formulation is achieved.

Therefore, the present invention refers to the use of amines and amides for the stabilization of a cosmetic composition comprising

(a) an organic micronized UV absorber selected from a benzophenone derivative and
(b) a dibenzoylmethane derivative.

The benzophenone UV absorbers preferably correspond to formula

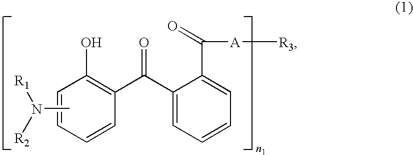

wherein
R₁ and R₂ independently from each other are; C₁-C₂₀alkyl; C₂-C₂₀alkenyl; C₃-C₁₀cycloalkyl; C₃-C₁₀cycloalkenyl; or R₁ and R₂ together with the linking nitrogen atom form a 5- or 6-membered heterocyclic ring;
n₁ is a number from 1 to 4;
when n₁=1,
R₃ is a saturated or unsaturated heterocyclic radical; hydroxy-C₁-C₅alkyl; cyclohexyl optionally substituted with one or more C₁-C₅alkyl; phenyl optionally substituted with a heterocyclic radical, aminocarbonyl or C₁-C₅alkylcarboxy;
when n₁ is 2,
R₃ is an alkylene-, cycloalkylene alkenylene or phenylene radical which is optionally interrupted by —O—; —NH—; a carbonyl- or carboxy group; a radical of formula *—CH₂—C≡C—CH₂—* or R₃ together with A forms a bivalent radical of the formula

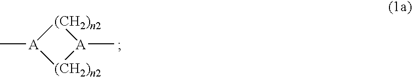

wherein
n₂ is a number from 1 to 3;
when n₁ is 3,
R₃ is an alkantriyl radical;
when n₁ is 4,
R₃ is an alkanetetrayl radical;
A is —O—; or —N(R₅)—; and
R₅ is hydrogen; C₁-C₅alkyl; or hydroxy-C₁-C₅alkyl.

C₁-C₂₀alkyl denotes a linear or branched, unsubstituted or substituted alkyl group such as, for example, methyl, ethyl, propyl, isopropyl, n-butyl, n-hexyl, cyclohexyl, n-decyl, n-dodecyl, noctadecyl, eicosyl, methoxyethyl, ethoxypropyl, 2-ethylhexyl, hydroxyethyl, chloropropyl, N,N-diethylaminopropyl, cyanoethyl, phenethyl, benzyl, p-tert-butylphenethyl, p-tert-octylphenoxyethyl, 3-(2,4-di-tert-amylphenoxy)-propyl, ethoxycarbonylmethyl-2-(2-hydroxyethoxy)ethyl or 2-furylethyl.

C₂-C₂₀alkenyl is for example allyl, methallyl, isopropenyl, 2-butenyl, 3-butenyl, isobutenyl, n-penta-2,4-dienyl, 3-methyl-but-2-enyl, n-oct-2-enyl, n-dodec-2-enyl, iso-dodecenyl, n-dodec-2-enyl or n-octadec-4-enyl.

C₃-C₁₀cycloalkyl is for example cyclopropyl, cyclobutyl, cyclopentyl, cycloheptyl, cyclooctyl, cyclononyl or cyclodecyl and preferably cyclohexyl. These radicals may be substituted, for example by one or more or equal or different C₁-C₄alkyl radicals, preferably by methyl, and/or hydroxy. If cycloalkyl radicals are substituted by one or more radicals, they are preferably substituted by one, two or four, preferably by one or two equal or radicals.

C₃-C₁₀cycloalkenyl is for example cyclopropenyl, cyclobutenyl, cyclopentenyl, cycloheptenyl, cycloocentyl, cyclononenyl or cyclodecenyl and preferably cyclohexenyl. These radicals may be substituted with one or more equal or different C₁-C₄alkyl radical, preferably with methyl, and/or hydroxy. If cycloalkenyl radicals are substituted with one or more radicals they are preferably substituted with one, two, three or four, preferably with one or two equal or different radicals.

Hydroxy-substituted C₁-C₅alkyl groups are for example hydroxymethyl, hydroxyethyl, hydroxypropyl, hydroxybutyl or hydroxypentyl.

An alkylene radical is preferably a C₁-C₁₂alkylene radical, like for example methylene, ethylene, propylene, butylene, hexylene or octylene.

The alkylene radicals may optionally be substituted by one or more C₁-C₅alkyl radicals.

If R₁ and R₂ are heterocyclic radicals, these comprise one, two, three or four equal or different ring hetero atoms. Special preference is given to heterocycles which contain one, two or three, especially one or two, identical or different hetero atoms. The heterocycles may be mono- or poly-cyclic, for example mono-, bi- or tri-cyclic. They are preferably mono- or bicyclic, especially monocyclic. The rings preferably contain 5, 6 or 7 ring members. Examples of monocyclic and bicyclic heterocyclic systems from which radicals occurring in the compounds of formula (1) or (2) may be derived are, for example, pyrrol, furan, thiophene, imidazole, pyrazole, 1,2,3-triazole, 1,2,4-triazole, pyridine, pyridazine, pyrimidine, pyrazine, pyran, thiopyran, 1,4-dioxane, 1,2-oxazine, 1,3-oxazine, 1,4-oxazine, indole, benzothiophene, benzofuran, pyrrolidine, piperidine, piperazine, morpholine and thiomorpholine.

Preference is given to compounds of formula (1), wherein
R₁ and R₂ independently from each other are hydrogen; C₁-C₂₀alkyl; C₂-C₂₀alkenyl; C₃-C₁₀cycloalkyl; C₃-C₁₀cycloalkenyl; or R₁ and R₂ together with the linking nitrogen atom form a 5- or 6-membered heterocyclic ring;
n₁ is a number from 1 to 4;
when n₁ is 1,
R₃ is a saturated or unsaturated heterocyclic radical; hydroxy-C₁-C₅alkyl; Cyclohexyl substituted with one or more C₁-C₅alkyl;
when n₁ is 2,
R₃ is an alkylene-, cycloalkylene- or alkenylene radical which is optionally interrupted by a carbonyl- or carboxy group;
when n₁ is 3,
R₃ is an alkantriyl radical; when n₁ is 4,
R₃ is an alkanetetrayl radical;
A is —O—; or —N(R₅)—; and
R₅ is hydrogen; C₁-C₅alkyl; or hydroxy-C₁-C₅alkyl.

Of preferred interest are compounds of formula (1), wherein
R₁ and R₂ are C₁-C₂₀alkyl, preferably C₁-C₅alkyl; and most preferably ethyl.

Preferably R₁ and R₂ in formula (1) have the same definition.

If in formula (1) n₁ is 1, compounds are preferred, wherein
R₃ is a saturated or unsaturated heterocyclic radical, most preferably a saturated heterocyclic radical.

Among these compounds are those preferred, wherein
R₃ is a monocyclic radical of 5, 6 or 7 ring members with one or more heteroatoms, preferably wherein
R₃ is morpholinyl; piperazinyl; piperidyl; pyrazolidinyl; imidazolidinyl; or pyrrolidinyl.

When n₁ is 1 further compounds of formula (1) are of interest wherein
R₃ is an unsaturated heterocyclic radical, preferably a polycyclic radical.

Most preferred are compounds of formula (1), wherein
R₃ is a radical of formula number

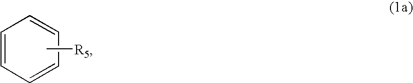

and
R₅ is polycyclic heteroaromatic radical with one or 2 heteroatoms.

Of preferred interest are compounds of formula (1), wherein
R₃ is a radical of formula

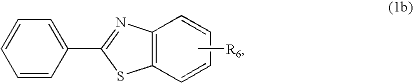

wherein
R₆ is hydrogen; or C₁-C₅alkyl.

If n₁ is 2,
R₃ is preferably a C₁-C₁₂alkylene radical, most preferably a C₂-C₈alkylene radical.

Mostly preferred are compounds of formula (1), wherein
R₃ is a radical of formula —CH₂—(CH₂)ₘ—CH₂—*;

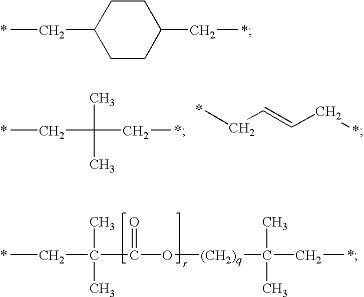

r is 0 or 1; and
q=is a number from 0 to 5.

If in formula (1) n₁ is 3,
R₃ is preferably a radical of formula

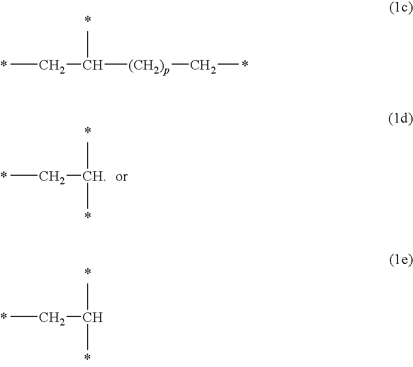

and
p is a number from 0 to 3.
R₁, R₂ and A are defined as in formula (1).

If in formula (1) n₁ is 4,
R₃ is a radical of formula

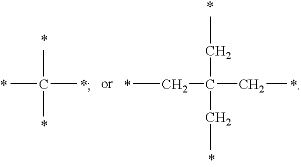

R₁, R₂ and A are defined as in formula (1).

Preferred compounds of the present invention correspond to formula

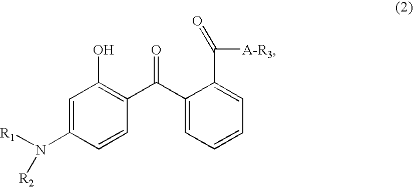

wherein
R₁ and R₂ independently from each other are hydrogen; or C₁-C₅alkyl;
A is —NH; or —O—; and
R₃ is a saturated or unsaturated heterocyclic radical.

Furthermore compounds of the present invention are preferred which correspond to formula

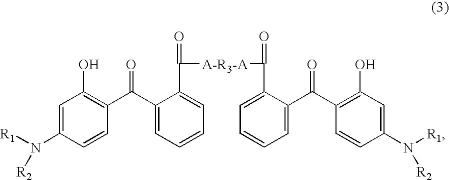

wherein
R₁ and R₂ independently from each other are hydrogen; or C₁-C₅alkyl;
A is —NH; or —O—; and
R₃ is a C₁-C₁₂alkylene radical; or R₃ together with A forms a bivalent radical of the formula

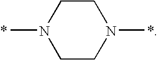

Preferred are also compounds of formula

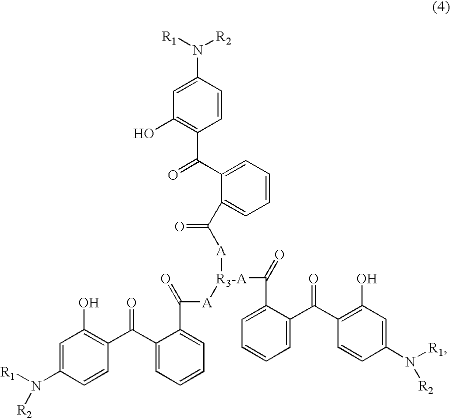

wherein
R₁ and R₂ independently from each other are hydrogen; or C₁-C₅alkyl;
A is —NH; or —O—;
R₃ is

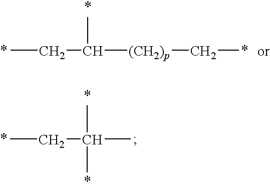

and
p is a number from 0 to 3.

Furthermore, compounds of formula

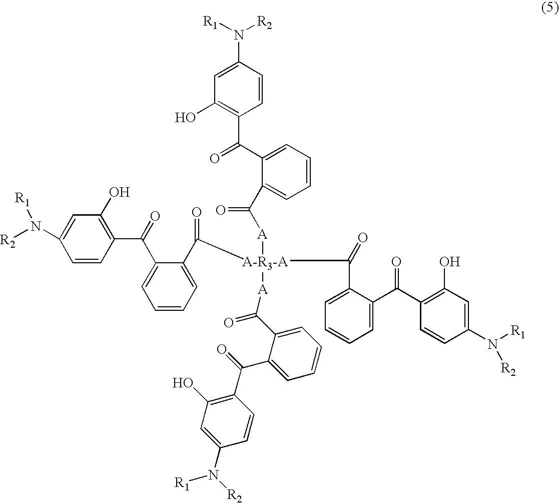

are preferred, wherein
R₃ is a radical of formula

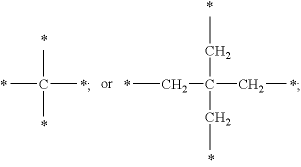

and
R₁, R₂ and A are defined as in formula (1).

Exemplified compounds of the present invention are of formulae

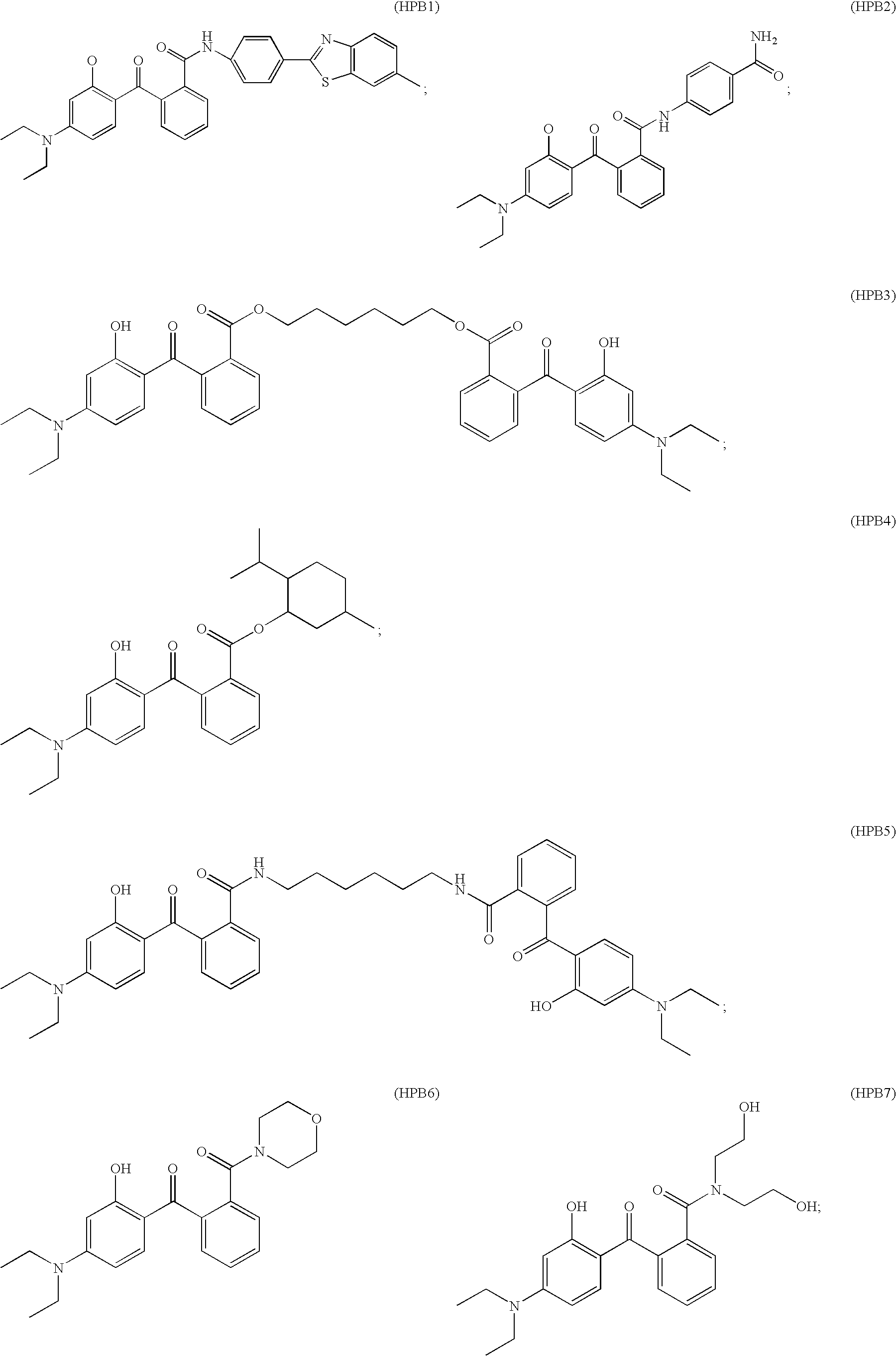

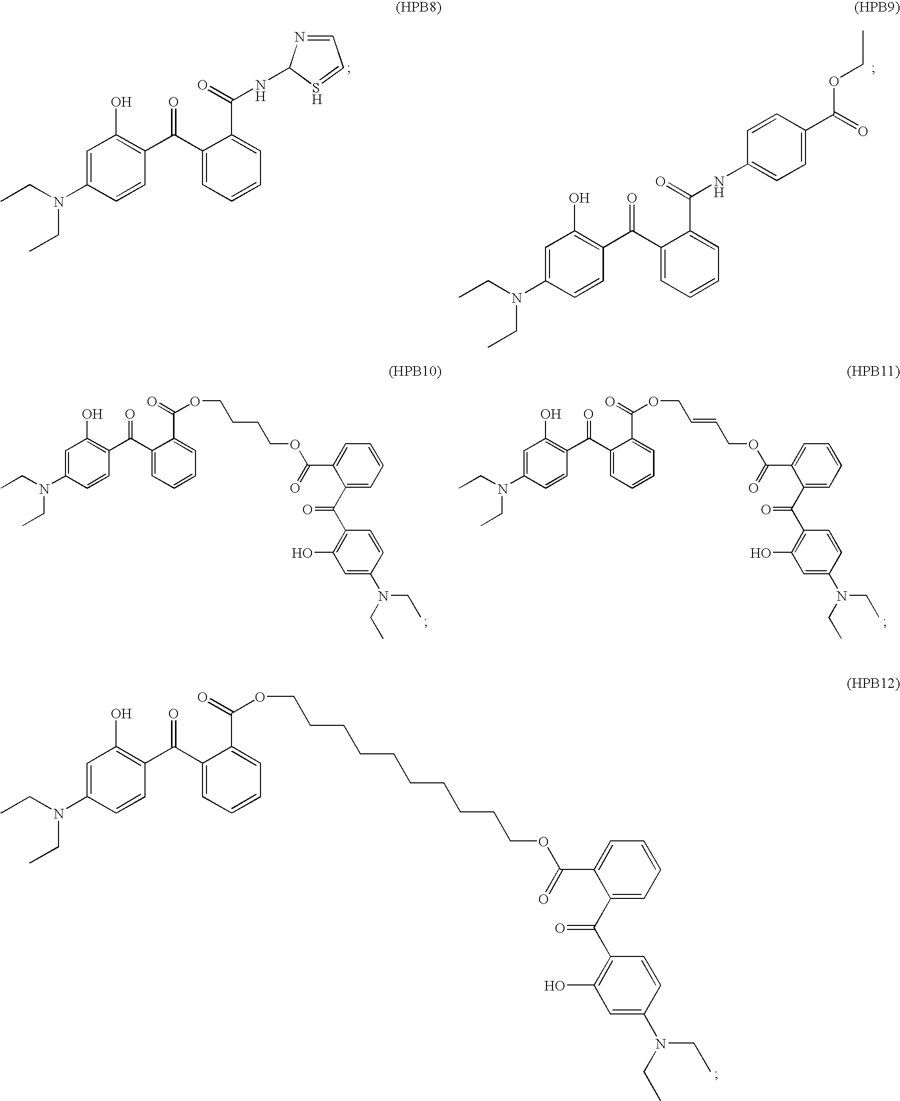

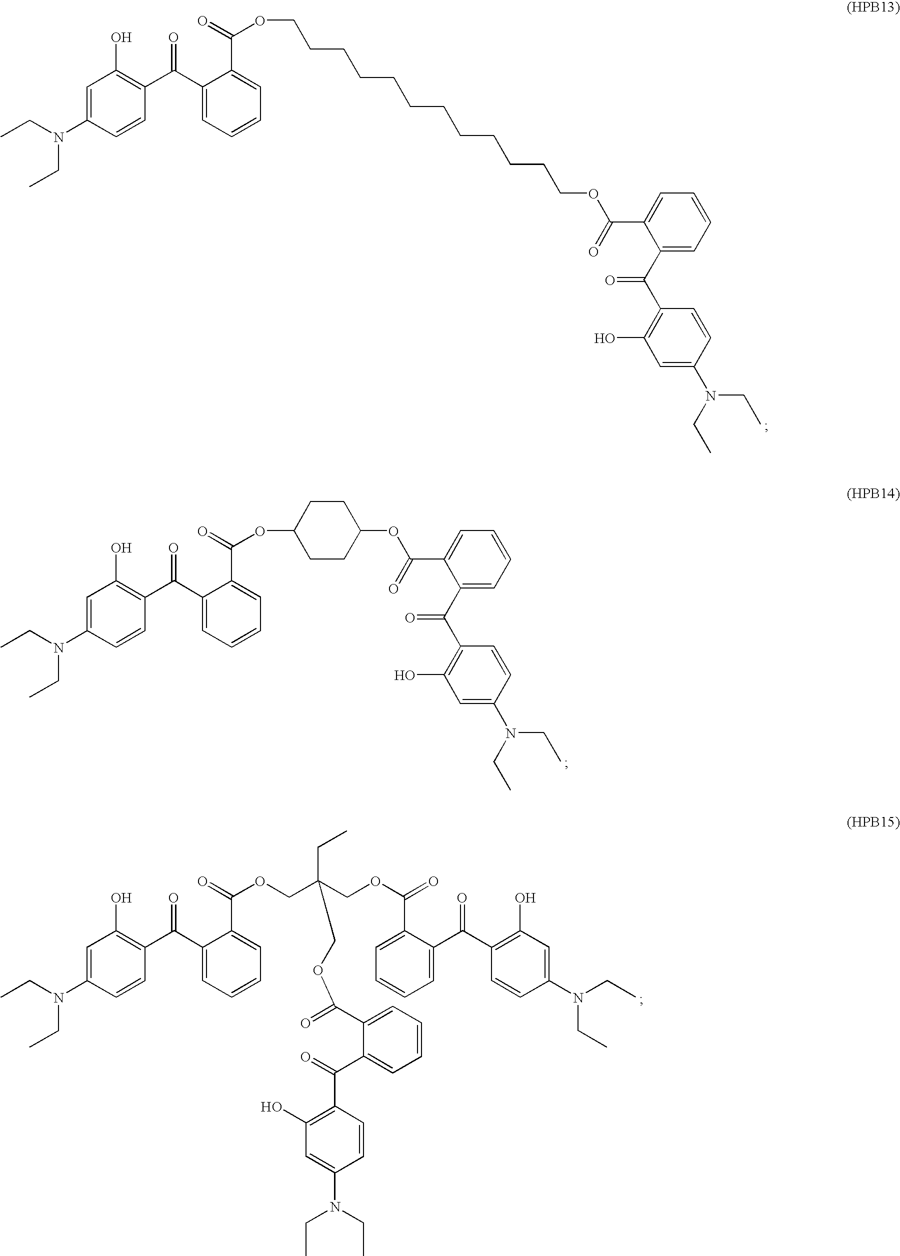

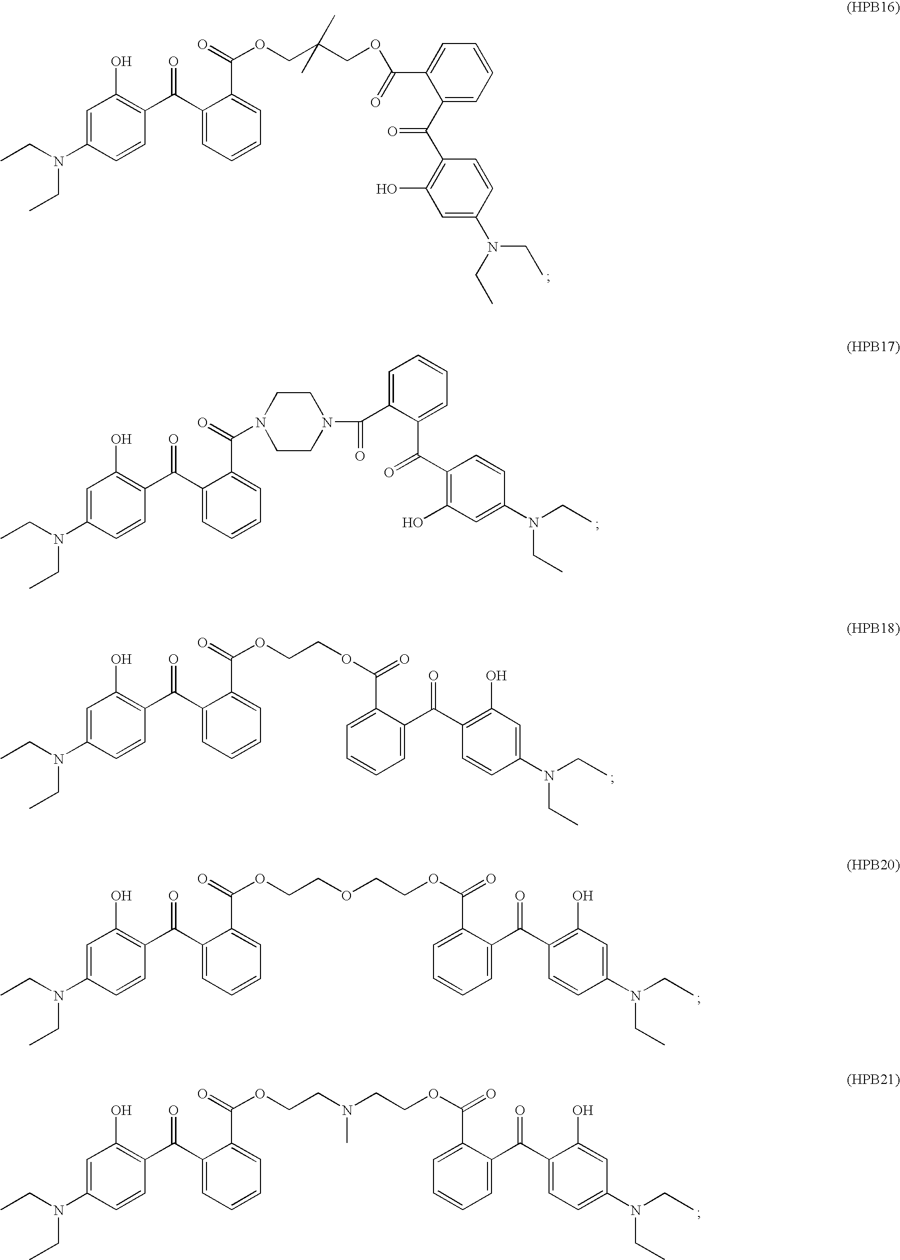

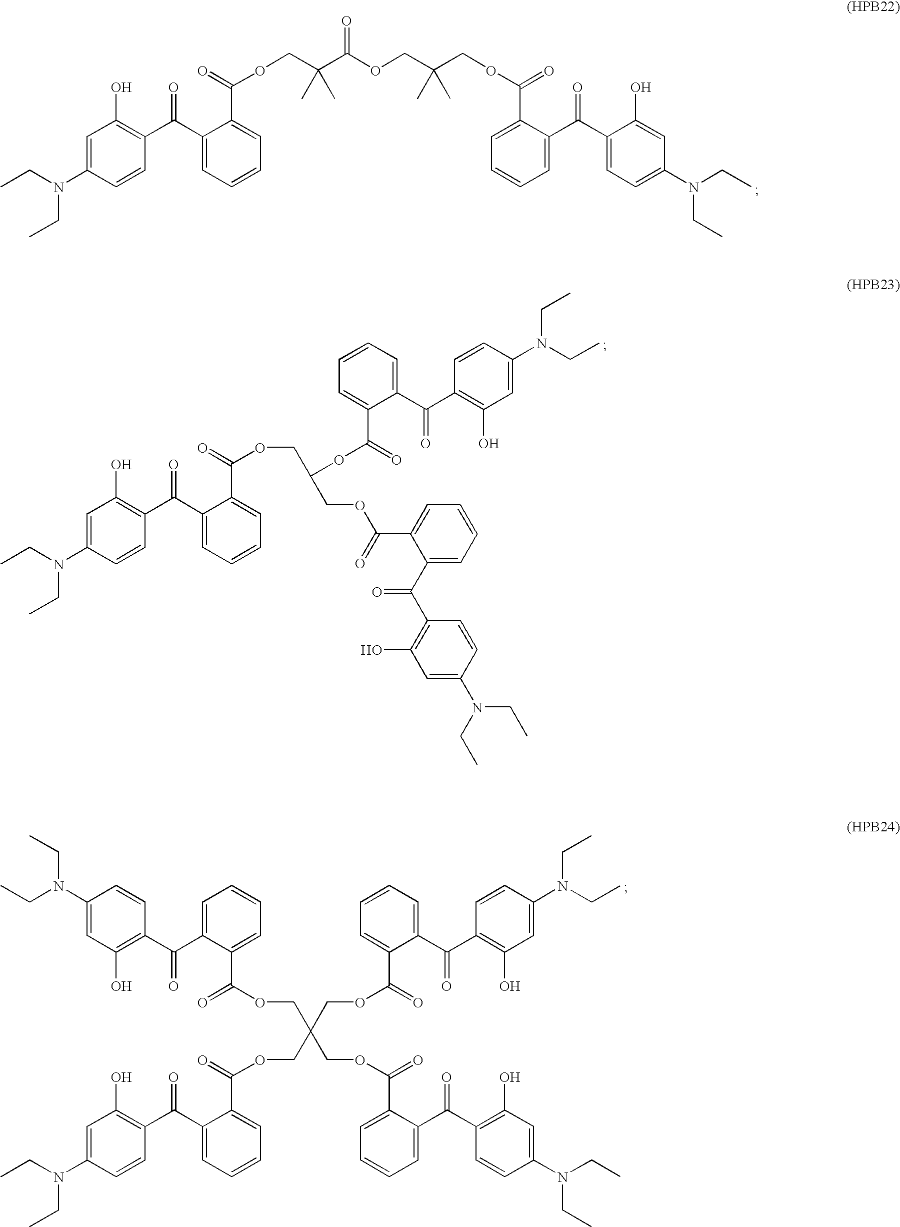

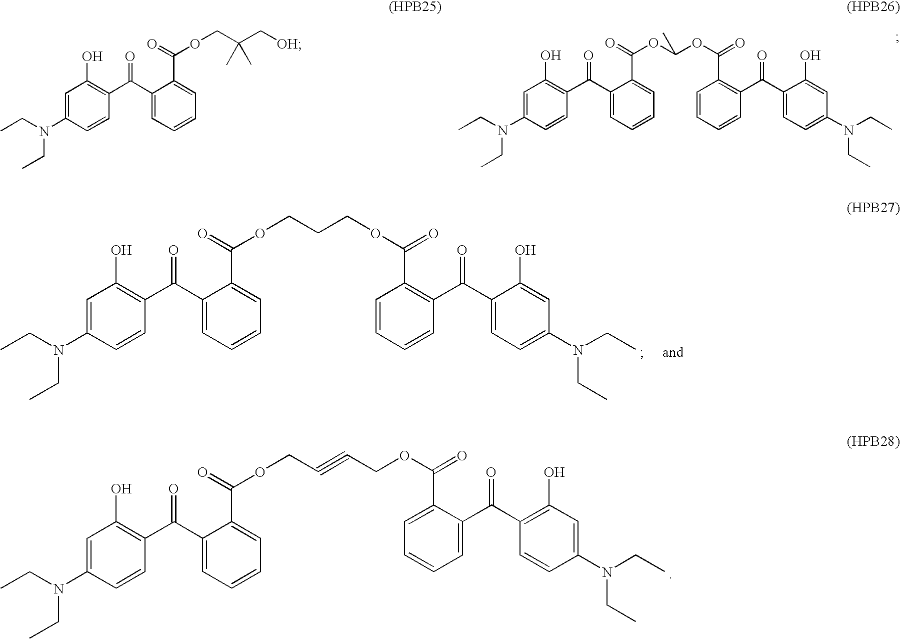

The compounds of formula (1) may be prepared according to known methods as described for example in EP-1,046,391.

Sparingly soluble organic compounds which are used in the present invention are present in the micronized state. They may be prepared by any known process suitable for the preparation of microparticles, for example wet-milling, wet-kneading, spray-drying from a suitable solvent, by expansion according to the RESS process (Rapid Expansion of Supercritical Solutions) of supercritical fluids (e.g. CO₂, by reprecipitation from suitable solvents, including supercritical fluids (GASR process=Gas Anti-Solvent Recrystallisation/PCA process=Precipitation with Compressed Anti-solvents).

As milling apparatus for the preparation of the sparingly soluble micronized organic compounds there may be used, for example, a jet mill, ball mill, vibratory mill or hammer mill, preferably a high-speed mixing mill. Even more preferable mills are modern ball mills; manufacturers of these types of mill are, for example, Netzsch (LMZ mill), Drais (DCP-Viscoflow or Cosmo), Bühler AG (centrifugal mills) or Bachhofer.

Examples of kneading apparatus for the preparation of the micronized organic UV absorbers are typical sigma-blade batch kneaders but also serial batch kneaders (IKA-Werke) or continuous kneaders (Continua from Werner and Pfleiderer).

The grinding of the sparingly soluble organic compounds used in the present invention is preferably carried out with a grinding aid.

The dispersing agent is used as a low molecular weight grinding aid for all the above micronization processes.

Useful anionic, non-ionic or amphoteric surfactants are disclosed below in the sections entitled “specific dispersing agents”.

Preferred useful grinding aids for an aqueous dispersion are anionic surfactants with a HLB (Hydrophile-Lipophile Balance) value higher than 8, more preferably higher than 10.

Any conventionally usable anionic, non-ionic or amphoteric surfactants can be used as dispersing agents. Such surfactant systems may comprise for example: carboxylic acids and their salts: alkaline soap of sodium, potassium and ammonium, metallic soap of calcium or magnesium, organic basis soap such as Lauric, myristic, palmitic, stearic and oleic acid etc., alkyl phosphates or phosphoric acid esters, acid phosphate, diethanolamine phosphate, potassium cetyl phosphate, ethoxylated carboxylic acids or polyethyleneglycol esters, PEG-n acylates. Fatty alcohol polyglycolether such as laureth-n, myreth-n, ceteareth-n, steareth-n, oleth-n. fatty acid polyglycolether such as PEG-n stearate, PEG-n oleate, PEG-n cocoate, monoglycerides and polyol esters, C₁₂-C₂₂ fatty acid mono- and di-esters of addition products of from 1 to 100 mol of ethylene oxide with polyols, fatty acid and polyglycerol ester such as monostearate glycerol, diisostearoyl polyglyceryl-3-diisostearates, polyglyceryl-3-diisostearates, triglyceryl diisostearates, polyglyceryl-2-sesquiisostearates or polyglyceryl dimerates. Mixtures of compounds from a plurality of those substance classes are also suitable. Fatty acid polyglycolesters such as monostearate diethylene glycol, fatty acid and polyethylene glycol esters, fatty acid and saccharose esters such as sucro esters, glycerol and saccharose esters such as sucro glycerides. Sorbitol and sorbitan, sorbitan mono- and diesters of saturated and unsaturated fatty acids having from 6 to 22 carbon atoms and ethylene oxide addition products, polysorbate-n series, sorbitan esters such as sesquiisostearate, sorbitan, PEG-(6)-isostearate sorbitan, PEG-(10)-sorbitan laurate, PEG-17-dioleate sorbitan, glucose derivatives, C8-C22 alkyl-mono and oligo-glycosides and ethoxylated analogues with glucose being preferred as the sugar component, O/W emulsifiers such as methyl gluceth-20 sesquistearate, sorbitan stearate/sucrose cocoate, methyl glucose sesquistearate, cetearyl alcohol/cetearyl glucoside, W/O emulsifiers such as methyl glucose dioleate/methyl glucose isostearate. Sulfates and sulfonated derivatives, dialkylsulfosuccinates, dioctyl succinate, alkyl lauryl sulfonate, linear sulfonated paraffins, sulfonated tetrapropylene sulfonate, sodium lauryl sulfates, ammonium and ethanolamine lauryl sulfates, lauryl ether sulfates, sodium laureth sulfates [Texapon N70] or sodium myreth sulfates [Texapon K14S], sulfosuccinates, acetyl isothionates, alkanolamide sulfates, taurines, methyl taurines, imidazole sulfates. Zwitterionic or amphoteric surfactants that carry at least one quaternary ammonium group and at least one carboxylate and/or sulfonate group in the molecule. Zwitterionic surfactants that are especially suitable are betaines, such as N-alkyl-N,N-dimethylammonium glycinates, cocoalkyldimethylammonium glycinate, N-acylaminopropyl-N,N-dimethylammonium glycinates, cocoacylaminopropyldimethylammonium glycinate and 2-alkyl-3-carboxymethyl-3-hydroxyethylimidazolines each having from 8 to 18 carbon atoms in the alkyl or acyl group and also cocoacylaminoethylhydroxyethylcarboxymethylglycinate, N-alkylbetaine, N-alkylaminobetaines.

Examples of suitable mild surfactants as dispersing agents, that is to say surfactants especially well tolerated by the skin, include fatty alcohol polyglycol ether sulfates, monoglyceride sulfates, mono- and/or di-alkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines and/or protein fatty acid condensation products, the latter preferably being based on wheat proteins.

Non ionic surfactants such as PEG-6 beeswax (and) PEG-6 stearate (and) polyglyceryl-2-isostearate [Apifac], glyceryl stearate (and) PEG-100 stearate. [Arlacel 165], PEG-5 glyceryl stearate [arlatone 983S], sorbitan oleate (and) polyglyceryl-3 ricinoleate. [Arlacel 1689], sorbitan stearate and sucrose cocoate [arlatone 2121], glyceryl stearate and laureth-23 [Cerasynth 945], cetearyl alcohol and ceteth-20 [Cetomacrogol Wax], cetearyl alcohol and colysorbate 60 and PEG-150 and stearate-20[Polawax GP 200, Polawax NF], cetearyl alcohol and cetearyl polyglucoside [Emulgade PL 1618], cetearyl alcohol and ceteareth-20 [Emulgade 1000NI, Cosmowax], cetearyl alcohol and PEG-40 castor oil [Emulgade F Special], cetearyl alcohol and PEG-40 castor oil and sodium cetearyl sulfate [Emulgade F], stearyl alcohol and steareth-7 and steareth-10 [Emulgator E 2155], cetearyl alcohol and szeareth-7 and steareth-10 [Emulsifying wax U.S.N.F], glyceryl stearate and PEG-75 stearate [Gelot 64], propylene glycol ceteth-3 acetate. [Hetester PCS], propylene glycol isoceth-3 acetate [Hetester PHA], cetearyl alcohol and ceteth-12 and oleth-12 [Lanbritol Wax N 21], PEG-6 stearate and PEG-32 stearate [Tefose 1500], PEG-6 stearate and ceteth-20 and steareth-20 [Tefose 2000], PEG-6 stearate and ceteth-20 and glyceryl stearate and steareth-20 [Tefose 2561], glyceryl stearate and ceteareth-20 [Teginacid H, C, X].

Anionic emulsifiers such as PEG-2 stearate SE, glyceryl stearate SE [Monelgine, Cutina KD], propylene glycol stearate [Tegin P], cetearyl Alcohol and Sodium cetearyl sulfate [Lanette N, Cutina LE, Crodacol GP], cetearyl alcohol and sodium lauryl sulfate [Lanette W], trilaneth-4 phopshate and glycol stearate and PEG-2 stearate [Sedefos 75], glyceryl stearate and sodium lauryl Sulfate [Teginacid Special]. Cationic acid bases such as cetearyl alcohol and cetrimonium bromide.

Most preferred dispersing agents are sodium alkyl sulfates or sodium alkyl ether sulfates, such as sodium laureth sulfate [Texapon N70 from Cognis] or sodium myreth sulfate [Texapon K14 S from Cognis].

The specific dispersing agents may be used in an amount of, for example, from 1 to 30% by weight, especially from 2 to 20% by weight and preferably from 3 to 10% by weight, based on the total weight of the composition.

Useful solvents are water, brine, (poly-)ethylene glycol, glycerol or cosmetically acceptable oils. Other useful solvents are disclosed below in the sections entitled “Esters of fatty acids”, “Natural and synthetic triglycerides, including glyceryl esters and derivatives”, “Pearlescent waxes”, “Hydrocarbon oils” and “Silicones or siloxanes”.

The micronized sparingly soluble organic compounds so obtained usually have an average particle size from 0.02 to 2 micrometres, preferably from 0.03 to 1.5 micrometres and more especially from 0.05 to 1.0 micrometres.

The aqueous dispersion used in the present invention generally comprises

30-60, preferably 35 to 55 parts of the sparingly soluble organic micronized substance;
2-20, preferably 2 to 20 parts of the dispersing agent;
0.1-1 part, preferably 0.1 to 0.5 parts of a thickening agent (for example xanthan gum); and
20-68 parts of water;

The cosmetic formulations or pharmaceutical compositions according to the present invention can also comprise one or more than one further UV filter as listed in Table 1:

TABLE 1
Suitable UV filter substances and adjuvants which can be additionally used with the
UV absorbers according to the present invention

| No. | Chemical Name | CAS No. |
|---|---|---|
| 1 | (+/−)-1,7,7-trimethyl-3-[(4-methylphenyl)methylene]bicyclo- | 36861-47-9 |
| | [2.2.1]heptan-2-one; p-methyl benzylidene camphor | |
| 2 | 1,7,7-trimethyl-3-(phenylmethylene)bicyclo[2.2.1]heptan-2-one; ben- | 15087-24-8 |
| | zylidene camphor | |
| 3 | (2-Hydroxy-4-methoxyphenyl)(4-methylphenyl)methanone | 1641-17-4 |
| 4 | 2,4-dihydroxybenzophenone | 131-56-6 |
| 5 | 2,2′,4,4′-tetrahydroxybenzophenone | 131-55-5 |
| 6 | 2-Hydroxy-4-methoxy benzophenone; | 131-57-7 |
| 7 | 2-Hydroxy-4-methoxy benzophenone-5-sulfonic acid | 4065-45-6 |
| 8 | 2,2′-dihydroxy-4,4′-dimethoxybenzophenone | 131-54-4 |
| 9 | 2,2′-Dihydroxy-4-methoxybenzophenone | 131-53-3 |
| 10 | Alpha-(2-oxoborn-3-ylidene)toluene-4-sulphonic acid and its salts | 56039-58-8 |
| | (Mexoryl SL) | |
| 11 | 1-[4-(1,1-dimethylethyl)phenyl]-3-(4-methoxyphenyl)propane-1,3- | 70356-09-1 |
| | dione (Avobenzone) | |
| 12 | Methyl N,N,N-trimethyl-4-[(4,7,7-trimethyl-3-oxobicyclo[2,2,1]hept-2- | 52793-97-2 |
| | ylidene)methyl]anilinium sulphate (Mexoryl SO) | |
| 22 | 3,3,5-Trimethyl cyclohexyl-2-hydroxy benzoate; homosalate | 118-56-9 |
| 23 | Isopentyl p-methoxycinnamate; isoamyl methoxy cinnamate | 71617-10-2 |
| 27 | menthyl-o-aminobenzoate | 134-09-8 |
| 28 | Menthyl salicylate | 89-46-3 |
| 29 | 2-Ethylhexyl 2-cyano,3,3-diphenylacrylate; octocrylene | 6197-30-4 |
| 30 | 2-ethylhexyl 4-(dimethylamino)benzoate | 21245-02-3 |
| 31 | 2-ethylhexyl 4-methoxycinnamate; octyl methoxy cinnamate | 5466-77-3 |
| 32 | 2-ethylhexyl salicylate | 118-60-5 |
| 33 | Benzoic acid,4,4′,4″-(1,3,5-triazine-2,4,6-triyltriimino)tris-, tris(2- | 88122-99-0 |
| | ethylhexyl)ester; 2,4,6-Triaznilino-(p-carbo-2′-ethylhexyl-1′-oxi)-1,3,5- | |
| | triazine (Octyl Triazone) | |
| 34 | 4-aminobenzoic acid | 150-13-0 |
| 35 | Benzoic acid, 4-amino-, ethyl ester, polymer with oxirane | 113010-52-9 |
| 38 | 2-phenyl-1H-benzimidazole-5-sulphonic acid; phenylbenzimida- | 27503-81-7 |
| | zole sulfonic acid | |
| 39 | 2-Propenamide, N-[[4-[(4,7,7-trimethyl-3-oxobicyclo[2.2.1]hept-2- | 147897-12-9 |
| | ylidene)methyl]phenyl]methyl]-, homopolymer | |
| 40 | Triethanolamine salicylate | 2174-16-5 |
| 41 | 3,3′-(1,4-phenylenedimethylene)bis[7,7-dimethyl-2-oxo-bicy- | 90457-82-2 |
| | clo[2.2.1]heptane-1 methanesulfonic acid] (Cibafast H) | |
| 42 | Titanium dioxide (primary particle size 10-50 nm) | 13463-67-7 |
| | For example T805 or Eusolex T-AVO, Eusolex T-2000, Titaniumdi- | |
| | oxid VT 817 | |
| 44 | Zinc oxide (primary particle size 20-100 nm) | 1314-13-2 |
| | For example Zinc oxide NDM, Zinc oxide Z-Cote HP1, Nanox Zinc | |
| | oxide | |
| 45 | 2,2′-Methylene-bis-[6-(2H-benzotriazol-2-yl)-4-(1,1,3,3-tetramethyl- | 103597-45-1 |
| | butyl)-phenol] (Tinosorb M) | |
| 46 | 2,4-bis{[4-(2-ethylhexyloxy)-2-hydroxy]-phenyl}-6-(4-methoxyphe- | 187393-00-6 |
| | nyl)-(1,3,5)-triazine (Tinosorb S) | |
| 47 | 1H-Benzimidazole-4,6-disulfonic acid, 2,2′-(1,4-phenylene)bis-, di- | 180898-37-7 |
| | sodium salt | |
| 48 | Benzoic acid, 4,4′-[[6-[[4-[[(1,1-dimethylethyl)amino]carbonyl]- | 154702-15-5 |
| | phenyl]amino]1,3,5-triazine-2,4-diyl]diimino]bis-, bis(2-ethylhexyl)- | |
| | ester; diethylhexyl butamido triazone (Uvasorb HEB) | |
| 49 | Phenol, 2-(2H-benzotriazol-2-yl)-4-methyl-6-[2-methyl-3-[1,3,3,3- | 155633-54-8 |
| | tetramethyl-1-[(trimethylsilyl)oxy]disiloxanyl]propyl]-; drometrizole | |
| | trisiloxane (Mexoryl XL) | |
| 50 | Dimethicodiethylbenzalmalonate; Polysilicone 15 (Parsol SLX) | 207574-74-1 |
| 51 | Benzene sulfonic acid, 3-(2H-benzotriazol-2-yl)-4-hydroxy-5-(1- | 92484-48-5 |
| | methylpropyl)-, monosodium salt (Tinogard HS) | |
| 52 | 1-Dodecanaminium, N-[3-[[4-(dimethylamino)benzoyl]amino]propyl]- | 156679-41-3 |
| | N,N-dimethyl-, salt with 4-methylbenzenesulfonic acid (1:1) (Escalol | |
| | HP610) | |
| 53 | 1-Propanaminium, N,N,N-trimethyl-3-[(1-oxo-3-phenyl-2-propenyl)- | 177190-98-6 |
| | amino]-, chloride | |
| 54 | 1H-Benzimidazole-4,6-disulfonic acid, 2,2′-(1,4-phenylene)bis- | 170864-82-1 |
| 55 | 1,3,5-Triazine, 2,4,6-tris(4-methoxyphenyl)- | 7753-12-0 |
| 56 | 1,3,5-Triazine, 2,4,6-tris[4-[(2-ethylhexyl)oxy]phenyl]- | 208114-14-1 |
| 57 | 1-Propanaminium, 3-[[3-[3-(2H-benzotriazol-2-yl)-5-(1,1-dimethyl- | 340964-15-0 |
| | ethyl)-4-hydroxyphenyl]-1-oxopropyl]amino]-N,N-diethyl-N-methyl-, | |
| | methyl sulfate (salt) | |
| 58 | 2-Propenoic acid, 3-(1H-imidazol-4-yl)- | 104-98-3 |
| 59 | Benzoic acid, 2-hydroxy-, [4-(1-methylethyl)phenyl]methyl ester | 94134-93-7 |
| 60 | 1,2,3-Propanetriol, 1-(4-aminobenzoate) (Glyceryl PABA) | 136-44-7 |
| 61 | Benzene acetic acid, 3,4-dimethoxy-a-oxo- | 4732-70-1 |
| 62 | 2-Propenoic acid, 2-cyano-3,3-diphenyl-, ethyl ester | 5232-99-5 |
| 63 | Anthralinic acid, p-menth-3-yl ester | 134-09-8 |
| 64 | 2,2′-bis(1,4-phenylene)-1H-benzimidazole-4,6-disulphonic acid mo- | 349580-12-7, |
| | no sodium salt or Disodium phenyl dibenzimidazole tetrasulfonate | |
| | (Neo Heliopan AP) | |
| 65 | 1,3,5-Triazine-2,4,6-triamine, N,N′-bis[4-[5-(1,1-dimethylpropyl)-2- | 288254-16-0 |
| | benzoxazolyl]phenyl]-N″-(2-ethylhexyl)- (Uvasorb k2A) | |
| 66 | Merocyanine derivatives as described in WO 2004006878, | |
| | WO2006032741, IPCOM000022279D and in IP.COM JOURNAL | |
| | (2005), 5(7B), 18 | |
| 67 | 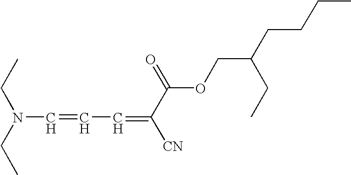 | |
| 68 | sterols (cholesterol, lanosterol, phytosterols), as described in | |
| | WO0341675 | |
| 69 | mycosporines and/or mycosporine-like amino acids as described in | |
| | WO2002039974, e.g. Helioguard 365 from Milbelle AG, isolated my- | |
| | cosporine like amino acids from the red alga porphyra umbilicalis | |
| | (INCI: Porphyra Umbilicalis) that are encapsulated into liposomes,) | |
| 70 | alpha-lipoc-acid as described in DE 10229995 | |
| 71 | synthetic organic polymers as described in EP 1371358, | |
| | [0033]-[0041] | |
| 72 | phyllosilicates as described in EP 1371357 [0034]-[0037] | |
| 73 | silica compounds as described in EP1371356, [0033]-[0041] | |
| 74 | inorganic particles as described in DE10138496 [0043]-[0055] | |
| 75 | latex particles as described in DE10138496 [0027]-[0040] | |
| 76 | 1H-Benzimidazole-4,6-disulfonic acid, 2,2′-(1,4-phenylene)bis-, | 180898-37-7 |
| | disodium salt; Bisimidazylate (Neo Heliopan APC) | |
| 77 | 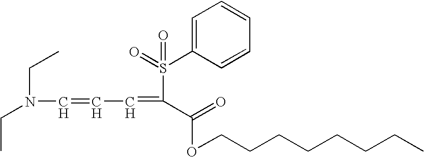 | |
| 78 | 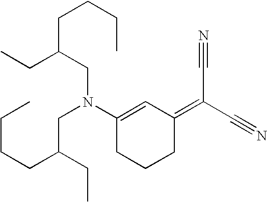 | |
| 79 | 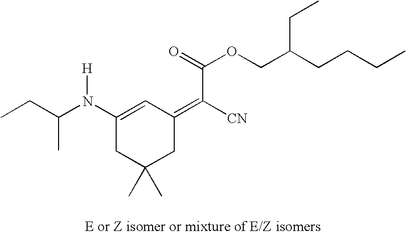 | |
| 80 | 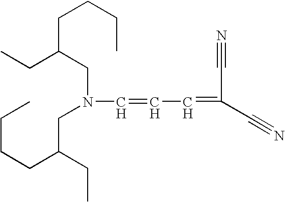 | |
| 81 | Di-2-ethylhexyl-3,5-dimethoxy-4-hydroxy-benzalmalonate (Oxynex | |
| | ST, EMD Chemicals, as described in US 20040247536) | |
| 82 | 2,4,6-Tris-1,1′,4′,1″-terphenyl-4-yl-1,3,5-triazine | |
| 83 | 2,4,6-Tris(p-biphenylyl)-s-triazine | 31274-51-8 |

Preferred dibenzoylmethane derivative used in the present invention is the 4-(tert-butyl)-4′-methoxydibenzoylmethane (also called avobenzone, CAS No. 70356-09-1), which is commercialised by DSM under the brand name Parsol® 1789 and Merck under the brand name Eusolex® 9020. Other dibenzoylmethane derivatives are described in U.S. Pa. Nos. 4,489,057, 4,387,089 and 4,562,067.

The amines as used in the present invention are preferably alkanolamines.

Examples of these compounds are 2-aminobutanol, 2-(2-aminoethoxy)ethanol, aminoethyl propanediol, aminomethyl propanediol, aminomethyl propanol (AMP), aminopropanediol, bis-hydroxyethyl tromethamine, butyl diethanolamine, butylethanolamine, DEA PG-oleate, dibutyl ethanolamine, diethanolamine, diethyl ethanolamine (DEA), dihydroxyethyl toluidine, diisopropanolamine, dimethylamino methylpropanol, dimethyl isopropanolamine, dimethyl mea, ethanolamine (EA), ethyl ethanolamine, hydroxyethyl palmityl oxyhydroxypropyl palmitamide, isopropanolamine, n-lauryl diethanolamine, MEA-sulfite, methylethanolamine (MEA), mixed isopropanolamines, PEG-2 tallowamide DEA, TEA-diricinoleate, TEA-lauryl ether, triethanolamine (TEA), triisopropanolamine, tromethamine (Tris Amino).

More preferred alkanolamines are aminomethyl propanol (AMP), ethanolamine (EA), diethyl ethanolamine (DEA), tromethamine (Tris Amino) and triethanolamine (TEA).

Further preferred amines are selected from alkoxylated amines which correspond to the formula

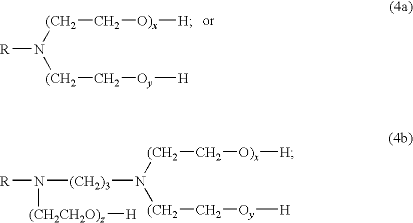

wherein
R is C₁-C₃₀alkyl; and
X, y and z independently from each other are a number from 1 to 20.

A typical representative of this class is PEG-10 Hydrogenated Tallow Amine.

Further preferred are amines are selected from amine oxides which correspond to the formula (6) RR′R″NO, wherein
R is C₁₂-C₃₀alkyl; and
R′ and R″ independently from each other are C₁-C₁₂alkyl.

A typical example is lauramine oxide, which chemically is N,N-dimethyl lauramine oxide.

Examples of amines that may be used in the present invention:

Acetyl carnitine HCl, adenine, adenosine, adenosine cyclic phosphate, almondamidopropyl dimethylamine, amidinoproline, amino bispropyl dimethicone, 5-amino-4-chloro-o-cresol, 5-amino-6-chloro-o-cresol, 2-amino-6-chloro-4-nitrophenol, 4-amino-m-cresol, 6-amino-m-cresol, 6-amino-o-cresol, 3-amino-2,4-dichlorophenol, 5-amino-2,6-dimethoxy-3-hydroxypyridine, 2-amino-4,5-dimethylthiazole HBr, aminoethanesulfinic acid, aminoethyl aminopropyl dimethicone, aminoethyl sulfate, 5-amino-4-fluoro-2-methylphenol sulfate, aminoguanidine bicarbonate, aminoguanidine HCL, 2-amino-4-hydroxyethylaminoanisole, 2-amino-4-hydroxyethylaminoanisole sulfate, 2-amino-3-hydroxypyridine, 4-amino-2-hydroxytoluene, 2-aminomethyl-p-aminophenol HCL, 4-amino-2-nitrodiphenylamine-2′-carboxylic acid, 2-amino-3-nitrophenol, 2-amino-4-nitrophenol, 2-amino-5-nitrophenol, 4-amino-2-nitrophenol, 4-amino-3-nitrophenol, 2-amino-4-nitrophenol sulfate, m-aminophenol HCL, p-aminophenol HCL, m-aminophenol, o-aminophenol, p-aminophenol, m-aminophenol sulfate, o-aminophenol sulfate, p-aminophenol sulfate, 3-aminopropane sulfonic acid, aminopropyl dehydrogenate phosphate, aminopropyl kojyl phosphate, aminopropyl methylenedioxyphenyl phosphate, aminopropyl tocopheryl phosphate, aminotriazine pentane carboxamide mipa, aminotrimethylene phosphonic acid, amodimethicone, aspartame, avocadamidopropyl dimethylamine, babassuamidopropyl dimethylamine, basic blue 3, basic red 118, behenamidopropyl dimethylamine, behenamidopropyl dimethylamine behenate, behenamidopropyl dimethylamine lactate, benzoguanamine, bisamino PEG/PPG-41/3 aminoethyl pg-propyl dimethicone, bis-aminopropyl dimethicone, bis(c13-15 alkoxy) pg-amodimethicone, 1,3-bis-(2,4-diaminophenoxy)propane, 4,6-bis(2-hydroxyethoxy)-m-phenylenediamine HCL, 2,6-bis(2-hydroxyethoxy)-3,5-pyridinediamine HCL, N,N′-bis(2-hydroxyethyl)-2-nitro-p-phenylenediamine, N,N-bis(2-hydroxyethyl)-p-phenylenediamine sulfate, brucine sulfate, calcium disodium EDTA, c12-14 alkyl diaminoethylglycine HCL, carnitine HCL, carnitine pica, carnosine, cetylamine hydrofluoride, cetyldimethylamine hydrolyzed hempseedate, chloramine t, chlorhexidine, 4-chloro-2-aminophenol, 2-chloro-6-ethylamino-4-nitrophenol, 2-chloro-5-nitro-N-hydroxyethyl p-phenylenediamine, 2-chloro-p-phenylenediamine, 2-chloro-p-phenylenediamine sulfate, cocamidopropyl dimethylamine, cocamidopropyl dimethylamine dihydroxy-methylpropionate, cocamidopropyl dimethylamine lactate, cocamidopropyl dimethylamine propionate, cocamidopropyl morpholine, cocamidopropyl morpholine lactate, cocamine, cocoyl polyglyceryl-4 hydroxypropyl dihydroxyethylamine, cyclohexylamine, N-cyclopentyl-m-aminophenol, cytosine, DEA-C₁₂₋₁₃ alkyl sulfate, DEA-C₈₋₁₈ perfluoroalkylethyl phosphate, DEA-PEG-4 laurate, DEA pg-propyl PEG/PPG-18/21 dimethicone, DEA-polyperfluoroethoxymethoxy PEG-2 phosphate, dicarboxy carnosine HCL, 2,4-diaminodiphenylamine, 4,4′-diaminodiphenylamine, 4,4′-diaminodiphenylamine sulfate, 2,4-diamino-5-methylphenetole HCL, 2,4-diamino-5-methylphenoxyethanol HCL, 4,5-diamino-1-methylpyrazole HCL, 2,4-diaminophenol, 2,4-diaminophenol HCL, 2,4-diaminophenoxyethanol HCL, 2,4-diaminophenoxyethanol sulfate, 2,6-diaminopyridine, 2,6-diaminopyridine sulfate, 2,6-diamino-3-((pyridin-3-yl)azo)pyridine, diaminopyrimidine oxide, diammonium EDTA, dibehenyl methylamine, dibromopropamidine diisethionate, dicocamine, dicocodimethylamine dilinoleate, diethanolamine bisulfate, diethylamine, diethylaminoethyl cocoate, diethylaminoethyl stearate, diethylamino hydroxybenzoyl hexyl benzoate, N,N-diethyl-m-aminophenol, N,N-diethyl-m-aminophenol sulfate, diethylenetriamine pentamethylene phosphonic acid, diethylhexylamine, N,N-diethyl-p-phenylenediamine sulfate, N,N-diethyltoluene-2,5-diamine HCL, dihydrogenated tallow methylamine, dihydroxyaluminum aminoacetate, dihydroxyethylamino hydroxypropyl oleate, 2,6-dihydroxyethylaminotoluene, dihydroxyethyl cocamine dioleate, dihydroxyethyl soyamine dioleate, dihydroxyethyl tallowamine dioleate, dihydroxyethyl tallowamine HCL, dihydroxyethyl tallowamine/ipdi copolymer, dihydroxyethyl tallowamine oleate, diisopropylamine, dilinoleamidopropyl dimethylamine, dimethicone propylethylenediamine behenate, 2,6-dimethoxy-3,5-pyridinediamine HCL, dimethoxysilyl ethylenediaminopropyl dimethicone, dimethylaminoethyl methacrylate, m-dimethylaminophenyl urea, dimethyl behenamine, dimethyl cocamine, dimethyl hydrogenated tallowamine, N,N′-dimethyl-n-hydroxyethyl-3-nitro-p-phenylenediamine, dimethyl lauramine, dimethyl lauramine dimer dilinoleate, dimethyl lauramine isostearate, dimethyl lauramine oleate, dimethyl myristamine, dimethyl palmitamine, 2,6-dimethyl-p-phenylenediamine HCL, N,N-dimethyl-p-phenylenediamine, 2,6-dimethyl-p-phenylenediamine, N,N-dimethyl-p-phenylenediamine sulfate, N,N-dimethyl 2,6-pyridinediamine HCL, dimethyl soyamine, dimethyl stearamine, dimethyl tallowamine, dimethyltolylamine, dioleoyl edetolmonium methosulfate, dipalmitamine, dipeptide diaminobutyroyl benzylamide diacetate, diphenhydramine HCL, dipropylenetriamine, disodium bisethylphenyl triaminotriazine stilbenedisulfonate, disodium cocaminopropyl iminodiacetate, disodium dicarboxyethyl cocopropylenediamine, disodium guanylate, disoyamidoethyl hydroxyethyl ammonium lactate, disoyamine, distearamidopropylmethylamine, distearyldimethylamine dilinoleate, ditallowamidoethyl hydroxypropylamine, ethanolamine HCL, 4-ethoxy-m-phenylenediamine sulfate, 3-ethylamino-p-cresol sulfate, 7-ethylbicyclooxazolidine, ethyl dimethylaminobenzoate, ethylhexyl bis-isopentylbenzoxazolylphenyl melamine, n-ethyl-3-nitro papa, fluorescent brightener 230, 4-fluoro-6-methyl-m-phenylenediamine sulfate, gerotine, glucamine, glucosamine, glucosamine ascorbate, glucosamine HCL, glucosamine sulfate, glucosamine thioctate, guanidine HCL, guanidine phosphate, HC blue no. 14, HC orange no. 5, HC red no. 14, HC red no. 15, HC yellow no. 14, HC yellow no. 15, hexamidine, hexamidine diisethionate, hexamidine diparabene, hexamidine paraben, histidine dna, hydrogenated ditallowamine, hydrogenated tallowamine, hydroxyethylaminomethyl-p-aminophenol HCL, 2-hydroxyethylamino-5-nitroanisole, hydroxyethyl carboxymethyl cocamidopropylamine, hydroxyethyl chitosan, 1-hydroxyethyl 4,5-diamino pyrazole sulfate, hydroxyethyl-2,6-dinitro-p-anisidine, hydroxyethyl-3,4-methylenedioxyaniline HCL, hydroxyethyl-2-nitro-p-toluidine, hydroxyethyl-p-phenylenediamine sulfate, 2-hydroxyethyl picramic acid, 4-hydroxypropylamino-3-nitrophenol, hydroxypropyl arginine lauryl/myristyl ether HCL, hydroxypropyl bis(n-hydroxyethyl-p-phenylenediamine) HCL, hydroxypropyl ethylenediamine carbomer, hydroxystearyl methylglucamine, imidazole, isopropylamine, n-isopropyl 4,5-diamino pyrazole sulfate, isostearamidopropyl dimethylamine, isostearamidopropyl dimethylamine gluconate, isostearamidopropyl dimethylamine glycolate, isostearamidopropyl dimethylamine lactate, isostearamidopropyl morpholine, isostearamidopropyl morpholine lactate, kinetin, lauramidobutyl guanidine acetate, lauramidobutyl guanidine HCL, lauramidopropyl dimethylamine, lauramidopropyl dimethylamine propionate, lauramine, lauraminopropylamine, lauriminodipropionic acid, laur/myrist/palmitamidobutyl guanidine acetate, lauroyl ethylenediamine triacetic acid, laurylamine dipropylenediamine, lauryl aminopropylglycine, lauryl diethylenediaminoglycine, lauryl diethylenediaminoglycine HCL, lauryl dimethylamine cyclocarboxypropyloleate, lidocaine HCL, linoleamidopropyl dimethylamine, linoleamidopropyl dimethylamine dimer dilinoleate, linoleamidopropyl diammonium lactate, lithium guanosine triphosphate, lysine azelate, lysine cocoate, mea-benzoate, mea-borate, mea-iodine, melamine peroxide, methenamine, methenammonium chloride, n-methoxyethyl-p-phenylenediamine HCL, 2-methoxymethyl-p-aminophenol HCL, 2-methoxy-p-phenylenediamine sulfate, 6-methoxy-2,3-pyridinediamine HCL, 4-methoxytoluene-2,5-diamine HCL, methyl aminomethylcyclohexane carboxamide HCL, 3-methylamino-4-nitrophenoxyethanol, p-methylaminophenol, p-methylaminophenol sulfate, methyl anthranilate, 4-methylbenzyl 4,5-diamino pyrazole sulfate, methyl dicocamine, 2,2′-methylenebis 4-aminophenol, 2,2′-methylenebis-4-aminophenol HCL, 3,4-methylenedioxyaniline, methylglucamine, 2-methyl-5-hydroxyethylaminophenol, n-methyl-3-nitro-p-phenylenediamine, methylthioadenosine, minkamidopropyl dimethylamine, mipa-borate, morpholine, myristamidobutyl guanidine acetate, myristamidopropyl dimethylamine, myristamidopropyl dimethylamine phosphate, myristaminopropionic acid, myrist/palmitamidobutyl guanidine acetate, 3-nitro-4-aminophenoxyethanol, 2-nitro-5-glyceryl methylaniline, 3-nitro-p-hydroxyethylaminophenol, 2-nitro-n-hydroxyethyl-p-anisidine, 4-nitrophenyl aminoethylurea, 4-nitro-o-phenylenediamine dihydrochloride, 2-nitro-p-phenylenediamine dihydrochloride, 4-nitro-ophenylenediamine HCL, 4-nitro-m-phenylenediamine, 4-nitro-o-phenylenediamine, 2-nitro-pphenylenediamine, 4-nitro-m-phenylenediamine sulfate, 4-nitro-o-phenylenediamine sulfate, 6-nitro-2,5-pyridinediamine, 6-nitro-o-toluidine, oatamidopropyl dimethylamine, olaflur, oleamidopropyl dimethylamine, oleamidopropyl dimethylamine glycolate, oleamidopropyl dimethylamine lactate, oleamidopropyl dimethylamine propionate, oleamine, olivamidopropyl dimethylamine, olivamidopropyl dimethylamine lactate, PABA, palmamine, palmitamidobutyl guanidine acetate, palmitamidopropyl diethylamine, palmitamidopropyl dimethylamine, palmitamidopropyl dimethylamine lactate, palmitamidopropyl dimethylamine propionate, palmitamine, palmitoleamidopropyl dimethylamine lactate, palmitoleamidopropyl dimethylamine propionate, PEG-3 2,2′-di-p-phenylenediamine, PEI-7, PEI-10, PEI-15, PEI-30, PEI-35, PEI-45, PEI-250, PEI-275, PEI-700, PEI-1000, PEI-1400, PEI-1500, PEI-1750, PEI-2500, PEI-14m, pentasodium aminotrimethylene phosphonate, pentasodium ethylenediamine tetramethylene phosphonate, pentasodium pentetate, pentetic acid, PGg-amodimethicone, pphenetidine, p-phenylenediamine HCL, m-phenylenediamine, p-phenylenediamine, m-phenylenediamine sulfate, p-phenylenediamine sulfate, n-phenyl-p-phenylenediamine HCL, n-phenyl-p-phenylenediamine, n-phenyl-p-phenylenediamine sulfate, phytosphingosine, phytosphingosine glycolate, phytosphingosine HCL, phytosphingosine lactate, phytosphingosine pica, polylysine HBR, potassium edtmp, potassium magnesium aspartate, pyridoxine hydroxybenzoate, quinine, ricinoleamidopropyl dimethylamine, ricinoleamidopropyl dimethylamine lactate, scordinine, sesamidopropyl dimethylamine, sodium bischlorophenyl sulfamine, sodium carboxyethyl tallow polypropylamine, sodium carboxymethyl cocopolypropylamine, sodium carboxymethyl tallow polypropylamine, sodium cocoyl methylaminopropionate, sodium diethylenetriamine pentamethylene phosphonate, sodium EDTMP, sodium guanosine cyclic monophosphate, sodium hydroxylauryldimonium ethyl phosphate, sodium lauroyl ethylenediamine triacetate, sodium lauryl diethylenediaminoglycinate, sodium picramate, sodium sulfanilate, soyamidopropyl dimethylamine, soyamine, soyaminopropylamine, spermidine HCL, sphinganine, starch diethylaminoethyl ether, stearamidoethyl diethanolamine, stearamidoethyl diethylamine, stearamidoethyl diethylamine phosphate, stearamidoethyl ethanolamine, stearamidoethyl ethanolamine phosphate, stearamidopropyl dimethylamine, stearamidopropyl dimethylamine lactate, stearamidopropyl dimethylamine stearate, stearamidopropyl morpholine, stearamidopropyl morpholine lactate, stearamine, stearoxypropyl dimethylamine, stearyl PG-dimethylamine, sunflowerseedamidopropyl dimethylamine, sunflowerseedamidopropyl dimethylamine lactate, sunflowerseedamidopropyl dimethylamine malate, sunflowerseedamidopropyl morpholine lactate, tallamidopropyl dimethylamine, tallowamidopropyl dimethylamine, tallow amine, tallowaminopropylamine, taurine, TEA-C₈₋₁₈ perfluoroalkylethyl phosphate, TEA-EDTA, TEA-hydrochloride, TEA-hydroiodide, TEA-lactate, TEA-lauroyl glutamate, TEA-pca, TEA-polyphosphate, TEA-salicylate, TEA-sorbate, TEA-sulfate, tetrahydroxyethyl ethylenediamine, tetrahydroxypropyl ethylenediamine, tetrahydroxypropyl ethylenediamine dioleate, tetrasodium EDTA, tetrasodium iminodisuccinate, thiamine diphosphate, thiamine HCL, thiamine nitrate, thiotaurine, tocopheryl dimethylglycinate HCL, tolnaftate, toluene-2,5-diamine, toluene-2,6-diamine, toluene-3,4-diamine, toluene-2,5-diamine sulfate, o-tolyl biguanide, 2,5,6-triamino-4-pyrimidinol sulfate, trilaurylamine, trimethylsiloxyamodimethicone, tri-PABA panthenol, tripotassium EDTA, trisodium dicarboxymethyl alaninate, trisodium EDTA, trisodium ethylenediamine disuccinate, trisodium hEDTA, trisodium NTA, tris-oleoyltromethamine ethane sulfate, tromethamine magnesium aluminum silicate, tyrosyl histidine HCL, uracil, wheat germamidopropyl dimethylamine, wheat germamidopropyl dimethylamine lactate, zinc magnesium aspartate.

Preferred amides are compounds which correspond to the formula (7) RCO—NXY, wherein
RCO— is a carboxylic acid or a fatty acid radical; and
X and Y independently from each other are hydrogen; or any alkyl or aromatic radical.

The amide class may be part of a heterocyclic ring, as in PCA.

R can also represent a nitrogen atom, as in Urea. Amides are hydrolyzable derivatives of carboxylic acids and are prepared via a number of synthetic and biological routes. A typical representative includes Cocamide.

Examples of amides as used in the present invention are acetamidocaproic acid, acetaminophen, acetaminosalol, acetanilid, acetyl arginine, acetylated cetyl hydroxyprolinate, n-acetyl dihydrosphingosine, acetyl glucosamine, acetyl glutamic acid, acetyl hydroxyproline, acetyl methionine, acetylphytosphingosine, acetyl trifluoromethylphenyl valylglycine, adamantanylcarboxamido hydroxylbenzamide, adamantanyl hydroxylterephthalamide, adamantylcarboxamido methylhydroxylbenzamide, adamantyl dihydroxybenzamide, adamantyl methylhydroxylterephthalamide, adamantyl trihydroxybenzamide, adipic acid dihydrazide, algaeoyl phytosphingosine, allantoin panthenol, aluminum hydrogenated tallow glutamate, aluminum PCA, aluminum stearoyl glutamate, aminopropyl laurylglutamine, ammonium polyacryloyldimethyl taurate, anserine, aspartame, behenamide, benzisothiazolinone, benzylidenecamphor hydrolyzed collagen sulfonamide, bis(C₁₃₋₁₅alkoxy)hydroxybutamidoamodimethicone, bis-hydroxyethyl biscetyl malonamide, bis-hydroxyethyl tocopherylsuccinoylamido hydroxypropane, bis-methoxypropylamido isodocosane, t-butylbenzamido hydroxylbenzamide, t-butylbenzamido hydroxylphenylacetamide, t-butylbenzamido methylhydroxylbenzamide, butyl resorcinol bis-succinoylphytosphingosine, O₁₆₋₂₂ acid amide MEA, calcium pantetheine sulfonate, calcium saccharin, caproyl sphingosine, caproyl tyrosine, capryloyl glycine, capryltyrosinamide, caprylyl pyrrolidone, capsaicin, carboxymethyl chitosan myristamide, carboxymethyl chitosan succinamide, ceramide 1, ceramide 2, ceramide 3, ceramide 4, ceramide 5, ceramide 1 a, ceramide 6 II, cetyloxypropyl glyceryl methoxypropyl myristamide, cetyl-PG hydroxyethyl decanamide, cetyl-PG hydroxyethyl palmitamide, chitosan lauramide succinamide, chitosan succinamide, chloroacetamide, cholesteryl hexyl dicarbamate pullulan, cloflucarban, cocamide, cocamide methyl MEA, cocoyl glutamic acid, cocoyl methyl beta-alanine, coenzyme a, crotamiton, decarboxy carnosine HCl, DEDM hydantoin, DEDM hydantoin dilaurate, diazolidinyl urea, dibehenamidopropyldimethylamine dilinoleate, dibromocyanoacetamide, dibutyldecyl IPDI, dibutylhexyl IPDI, dibutyl lauroyl glutamide, dibutyloctyl IPDI, dicapryloyl cystine, didecyltetradecyl IPDI, diethyl acetyl aspartate, diethyl caprylamide, diethylhexyl butamido triazone, diethylhexyl IPDI, diethyl toluamide, Di-HEMA trimethylhexyl dicarbamate, dihexyldecyl IPDI, dihydrogenated tallow phthalic acid amide, dihydroxyisopropyl capryloylcaprylamide, dihydroxyisopropyl palmoylpalmamide, dilinoleic acid/ethylenediamine copolymer, dimer dilinoleamidopropyl dibetaine, dimethiconol panthenol, dimethoxybenzamido phenylhydroxylacetamide, m-dimethylaminophenyl urea, dimethyl capramide, dimethylol urea, dimethyl urea, dioctyldecyl IPDI, dioctyldodecyl IPDI, dioctyldodecyl stearoyl glutamate, Di-PEG-2 soyamine IPDI, dipeptide diaminobutyroyl benzylamide diacetate, disodium cocoyl glutamate, disodium N-lauroyl aspartate, disoyamidoethyl hydroxyethyl ammonium lactate, distearamidopropylmethylamine, disteareth-75 IPDI, disteareth-100 IPDI, distearyl phthalic acid amide, ditallowamidoethyl hydroxypropylmonium methosulfate, di-TEAcocamide diacetate, dithiodiethyl bis-dihydroxydimethylbutyramide, dithiodiethyl bis-lactamide, dithiodiethyl bis-salicylamide, dithiomethylbenzamide, DMDM hydantoin, DMHF, DM hydantoin, erucamide, ethylene dihydrogenated tallowamide, ethylene dilinoleamide, ethylene dioleamide, ethylene distearamide, ethylhexyl benzyldioxopiperazyl acetate, ethylhexyl dimethoxybenzylidene dioxoimidazolidine propionate, ethyl lauroyl arginate HCl, ethyl menthane carboxamide, ethyl tosylamide, fluorosalan, fluridil, glycolamide stearate, glycyl glycine, hexyldecyl myristoyl methylaminopropionate, hippuric acid, hyaluronic acid, hydrogenated tallow amide, hydroxycaproyl phytosphingosine, hydroxycapryloyl phytosphingosine, hydroxyethyl carboxymethyl cocamidopropylamine, hydroxyethyl urea, hydroxylauroyl phytosphingosine, hydroxymethoxybenzyl pelargonamide, hydroxypalmitoyl sphinganine, hydroxyphenyl dihydroxybenzamide, hydroxyproline palmitamide, ilomastat, imidazolidinyl urea, inulin lauryl carbamate, iodopropynyl butylcarbamate, isostearamidomorpholine stearate, isostearamidopropyl epoxypropylmorpholinium chloride, ketoconazole, laccaic acid, lactamide, lactoyl phytosphingosine, lauramide, lauramidobutyl guanidine acetate, lauramidobutyl guanidine HCl, laur/myrist/palmitamidobutyl guanidine acetate, lauroyl glutamic acid, lauroyl methyl beta-alanine, lauroyl methyl glucamide, laurylgluconamide palmitates, lauryl methylglucamide, lidocaine HCl, linoleamide, lysine PCA, MDM hydantoin, melatonin, methoxycinnamidopropyl O₁₈₋₂₂ alkyldimonium tosylate, methoxy PEG-450 amidoglutaroyl succinimide, methoxy PEG-450 amido hydroxysuccinimidyl succinamate, methoxypropylgluconamide, methyl acetamide, methyl aminomethylcyclohexane carboxamide HCl, methyl diisopropyl propionamide, milkamidopropyl amine oxide, milkamidopropyl betaine, minkamidopropyl betaine, myristamidobutyl guanidine acetate, myristoyl glutamic acid, myristoyl methyl beta-alanine, myristoyl/palmitoyl oxostearamide/arachamide MEA, myristoyl succinoyl atelocollagen, myrist/palmitamidobutyl guanidine acetate, myristyl-pg hydroxyethyl decanamide, neotame, niacinamide, niacinamide ascorbate, niacinamide glycolate, niacinamide hydroxycitrate, niacinamide lactate, niacinamide malate, niacinamide mandelate, niacinamide salicylate, niacinamide thioctate, nicotinamide adenine dinucleotide, nylon-611, nylon-611/dimethicone copolymer, octylisothiazolinone, oleamide, 2-oleamido-1,3-octadecanediol, oleoyl tyrosine, ° leyl palmitamide, palmitamide, palmitamidobutyl guanidine acetate, palmitamidohexadecanediol, palmitoyl glutamic acid, palmitoyl glycine, palmitoyl methoxytryptamine, palmitoyl myristyl serinate, palmitoyl pg-linoleamide, pantetheine sulfonate, pantethine, panthenol, panthenyl ethyl ether, panthenyl ethyl ether acetate, panthenyl ethyl ether benzoate, panthenyl hydroxypropyl steardimonium chloride, panthenyl triacetate, PCA, PCA dimethicone, PCA ethyl cocoyl arginate, palmitate, PEG-3 diethylenetriamine dipalmamide, PEG-2 dimeadowfoamamidoethylmonium methosulfate, PG-palmoylpalmamide, phenacetin, phthalimidoperoxycaproic acid, phytosphingosine acetamide, picolinamide, polyacrylamide, polyacrylamidomethyl benzylidene camphor, polyamide-1, polyamide-2, polyamide-3, polybeta-alanine, polyepsilon-lysine, polylysine, polymethacrylamide, polymethacryloyl lysine, polymethylglutamate, poly-p-phenylene terephthalamide, polyvinylcaprolactam, polyvinylformamide, potassium azeloyl diglycinate, potassium caproyl tyrosine, potassium cocoyl glutamate, potassium cocoyl glycinate, potassium cocoyl PCA, potassium cocoyl taurate, potassium methyl cocoyl taurate, potassium PCA, prolinamidoethyl imidazole, pyricarbate, quaternium-80, retinyl formyl aspartamate, riboflavin, riboflavin tetraacetate, saccharin, salicylamide, salicylhydroxamic acid, salicyloyl phytosphingosine, salnacedin, s-lactoylglutathione, sodium cocoyl glycinate, sodium diethylaminopropyl cocoaspartamide, sodium dilauramidoglutamide lysine, sodium dimer coenzyme a, sodium lauramido diacetate, sodium lauroyl ethylenediamine triacetate, sodium methyltaurine cocoyl methyltaurate, sodium palmitoyl proline, sodium palm kernelamphopropionate, sodium pantetheine sulfonate, sodium PCA, sodium PCA thylsilanol, Sodium PEG-6 cocamide carboxylate, sodium PEG-8 cocamide carboxylate, sodium PEG-3 lauramide carboxylate, sodium peg-4 lauramide carboxylate, sodium phenylacetyl glutamine, sodium phenylacetyl isoglutamine, sodium polyacryloyldimethyl taurate, sodium polygamma-glutamate, sodium polyglutamate, sodium stearyl phthalamate, sodium taurine cocoyl methyltaurate, sodium zinc histidine dithiooctanamide, stearamide, stearamidodihydroxyisobutyl stearate, stearic hydrazide, N-stearoyldihydrosphingosine, stearoyl glutamic acid, stearyl acetyl glutamate, stearyl erucamide, stearylgluconamide dilaurate, succinoyl ascorbate pentapeptide-6, succinoyl atelocollagen, succinoyl serum albumin, tallow amide, TDI/trimellitic anhydride copolymer, TEA-cocamide diacetate, TEA-hydrogenated tallowoyl glutamate, TEA-PCA, tetraacetylphytosphingosine, tetramethylolglycoluril, tetrasodium disuccinoyl cystine, thenoyl methionate, thiodiglycolamide, thymine, tococysteamide, tosylamide, triacetyl retinoyl phytosphingosine, triclocarban, trihydroxypalmitamidohydroxypropyl myristyl ether, tris(PEG-2 Phenylalanylcarboxamido) cyclohexane, tyrosyl Histidine HCl, undecylenoyl glycine, urea, urea Peroxide, yeast palmitate or zinc PCA.

Further preferred amides are selected from alkanolamides which correspond to formula

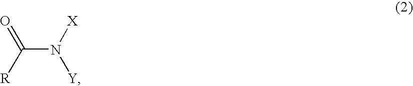

wherein
R is C₁-C₃₀alkyl;
X is the radical —CHR′CH₂OH;
R′ is hydrogen; or C₁-C₁₂alkyl; and
Y is —CHR′CH₂OH; C₁-C₁₂alkyl; or C₁-C₁₂hydroxyalkyl.

Examples of preferred alkanolamides are acetamide MEA, almondamide DEA, apricotamide DEA, avocadamide DEA, avocadamide DIPA, azelamide MEA, babassuamide DEA, babassuamide MEA, behenamide DEA, behenamide MEA, cCapramide DEA, cocamide DEA, cocamide DIPA, cocamide MEA, cocamide MIPA, cocoyl sarcosinamide DEA, cornamide/cocamide DEA, cornamide DEA, diethanolaminooleamide DEA, disodium cocamido MIPA PEG-4 sulfosuccinate, disodium lauramido MIPA, glycol sulfosuccinate, hydrogenated tallowamide DEA, hydroxycetyl hydroxyethylstearamide, hydroxyethyl ethylene dipalmitamide, hydroxyethyl stearamide-MIPA, hydroxylauroyl phytosphingosine, hydroxypropyl bisisostearamide MEA, hydroxypropyl bislauramide MEA, hydroxypropyl bispalmitamide MEA, hydroxystearamide MEA, isostearamide DEA, isostearamide MEA, isostearamide MIPA, lactamide MEA, lanolinamide DEA, lauramide DEA, lauramide MEA, lauramide MIPA, lauramide/Myristamide DEA, lauryl Malamide, lecithinamide DEA, linoleamide DEA, linoleamide MEA, linoleamide MIPA, minkamide DEA, myristamide DEA, myristamide MEA, myristamide MIPA, oatamide MEA, oleamide DEA, oleamide DIPA, oleamide MEA, oleamide MIPA, olivamide DEA, oliveamide MEA, palmamide DEA, palmamide MEA, palmamide MIPA, palmitamide DEA, palmitamide MEA, palm kernelamide DEA, palm kernelamide MEA, palm kernelamide MIPA, pantothenamide MEA, nutamide MEA, peanutamide MIPA, PEG-20 pocamide MEA, ricebranamide DEA, ricinoleamide DEA, ricinoleamide MEA, ricinoleamide MIPA, sesamide DEA, sesamide DIPA, soyamide DEA, stearamide AMP, stearamide DEA, stearamide DEA-distearate, stearamide DIBA-stearate, stearamide MEA, stearamide MEAstearate, stearamide MIPA, tallamide DEA, tallowamide DEA, tallowamide MEA, trideceth-2 Carboxamide MEA, undecylenamide DEA, undecylenamide MEA or wheat germamide DEA.

Preferred amides are also selected from alkoxylated amides which correspond to the formula

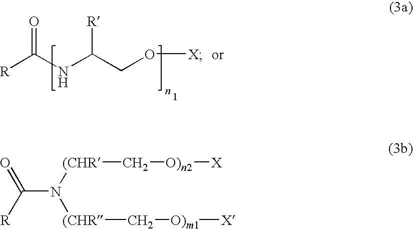

wherein
R is C₁-C₃₀alkyl;
R′ and R″ independently from each other are is hydrogen; or C₁-C₁₂alkyl;
X and X′ independently from each other are hydrogen; or or sulfosuccinyl;
n₁ is a number from 2 to 60;
n₂+m₁ independently from each other are a number from 1 to 60, and the sum of n₂+m₁≧3.

A typical representative of this group of compounds is PEG-7 Oleamide.

Further preferred amides are selected from alkylamido alkylamines which correspond to the formula

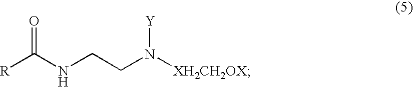

wherein
R is C₁-C₃₀alkyl;
X is hydrogen; or —CH₂COO—Na+ and
Y is —CH₂COO—Na+; —CH₂CH₂COO—Na+, —CH₂CHOHCH₂SO₃—Na+, or —CH₂CHOHCH₂OPO₃⁻ (H+/Na+).

The commercially available compounds in this class are fairly complicated mixtures of chemically related materials.

In a preferred embodiment of the present invention the amines are selected from mono-, di-, and triethanolamines, the micronized UV absorbers correspond to formula

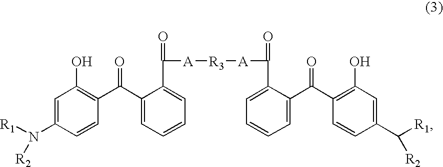

wherein
R₁ and R₂ independently from each other are hydrogen; or C₁-C₅alkyl;
A is —NH; or —O—; and
R₃ is a C₁-C₁₂alkylene radical; or R₃ together with A forms a bivalent radical of the formula)

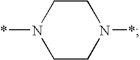

and the dibenzoylmethane derivative is 4-(tert-butyl)-4′-methoxydibenzoylmethane.

The present invention is also directed to cosmetic or pharmaceutical compositions, comprising

(a) 0.1 to 20% b.w. of a benzophenone UV absorber of formula (1);
(b) 0.1 to 10% b.w. of a methoxydibenzoylmethane derivative; and
(c) 0.1 to 50% b.w. of an amine or amide.

The cosmetic or pharmaceutical preparations can be prepared by physically mixing the UV absorber(s) with the adjuvant using customary methods, for example by simply stirring together the individual components, especially by making use of the dissolution properties of already known cosmetic UV absorbers, like octyl methoxy cinnamate, salicylic acid isooctyl ester, etc. The UV absorber can be used, for example, without further treatment, or in the micronized state, or in the form of a powder.

The amines and amides according to the present invention are useful for eliminating the conversion of larger scaled particles present in the composition.

Furthermore, the amines and amides according to the present invention are useful for eliminating the physic-chemical interactions with dibenzoylmethane derivatives.

The cosmetic composition according to the present invention is preferably used for preventing the human hair or skin from the harmful effect of UV radiation.

The cosmetic or pharmaceutical preparations may be, for example, creams, gels, lotions, alcoholic and aqueous/alcoholic solutions, emulsions, wax/fat compositions, stick preparations, powders or ointments. In addition to the above mentioned UV filters, the cosmetic or pharmaceutical preparations may contain further adjuvants as described below.

As water- and oil-containing emulsions (e.g. W/O, O/W, O/W/O and W/O/W emulsions or microemulsions) the preparations contain, for example, from 0.1 to 30% by weight, preferably from 0.1 to 15% by weight and especially from 0.5 to 10% by weight, based on the total weight of the composition, of one or more UV absorbers, from 1 to 60% by weight, especially from 5 to 50% by weight and preferably from 10 to 35% by weight, based on the total weight of the composition, of at least one oil component, from 0 to 30% by weight, especially from 1 to 30% by weight and preferably from 4 to 20% by weight, based on the total weight of the composition, of at least one emulsifier, from 10 to 90% by weight, especially from 30 to 90% by weight, based on the total weight of the composition, of water, and from 0 to 88.9% by weight, especially from 1 to 50% by weight, of further cosmetically acceptable adjuvants.

The cosmetic or pharmaceutical compositions/preparations according to the invention may also contain one or one more additional compounds as like fatty alcohols, esters of fatty acids, natural or synthetic triglycerides including glyceryl esters and derivatives, pearlescent waxes, hydrocarbon oils, silicones or siloxanes (organo-substituted polysiloxanes), fluorinated or perfluorinated oils, emulsifiers, adjuvants and additives, super-fatting agents, surfactants, consistency regulators/thickeners and rheology modifiers, polymers, biogenic active ingredients, deodorising active ingredients, anti-dandruff agents, antioxidants, hydrotropic agents, preservatives, bacteria-inhibiting agents, perfume oils, colorants, polymeric beads or hollow spheres as SPF enhancers.

### Cosmetic or Pharmaceutical Preparations

Cosmetic or pharmaceutical formulations are contained in a wide variety of cosmetic preparations. There come into consideration, for example, especially the following preparations: skin-care preparations, bath preparations, cosmetic personal care preparations, foot-care preparations, light-protective preparations, skin-tanning preparations, depigmenting prepartitions, insect-repellents, deodorants, antiperspirants, preparations for cleansing and caring for blemished skin, hair-removal preparations in chemical form (depilation), shaving preparations, fragrance preparations, cosmetic hair-treatment preparations,

### Presentation Forms

The final formulations listed may exist in a wide variety of presentation forms, for example:
in the form of liquid preparations as a W/O, O/W, O/W/O, W/O/W or PIT emulsion and all kinds of microemulsions,
in the form of a gel,
in the form of an oil, a cream, milk or lotion,
in the form of a powder, a lacquer, a tablet or make-up,
in the form of a stick,
in the form of a spray (spray with propellant gas or pump-action spray) or an aerosol, in the form of a foam, or
in the form of a paste.

Of special importance as cosmetic preparations for the skin are light-protective preparations, such as sun milks, lotions, creams, oils, sun blocks or tropicals, pretanning preparations or after-sun preparations, also skin-tanning preparations, for example self-tanning creams. Of particular interest are sun protection creams, sun protection lotions, sun protection milk and sun protection preparations in the form of a spray.

Of special importance as cosmetic preparations for the hair are the above-mentioned preparations for hair treatment, especially hair-washing preparations in the form of shampoos, hair conditioners, hair-care preparations, e.g. pre-treatment preparations, hair tonics, styling creams, styling gels, pomades, hair rinses, treatment packs, intensive hair treatments, hair-straightening preparations, liquid hair-setting preparations, hair foams and hairsprays. Of special interest are hair-washing preparations in the form of shampoos.

Other typical ingredients in such formulations are preservatives, bactericides and bacteriostatic agents, perfumes, dyes, pigments, thickening agents, moisturizing agents, humectants, fats, oils, waxes or other typical ingredients of cosmetic and personal care formulations such as alcohols, poly-alcohols, polymers, electrolytes, organic solvents, silicon derivatives, emollients, emulsifiers or emulsifying surfactants, surfactants, dispersing agents, antioxidants, anti-irritants and anti-inflammatory agents etc.

The cosmetic preparation according to the invention is distinguished by excellent protection of human skin against the damaging effect of sunlight.

### EXAMPLES

### B. Working Example

### Examples 1 to 6

| | | Ex. 1 | Ex. 2 | Ex. 3 | Ex. 4 | Ex. 5 | Ex. 6 |
|---|---|---|---|---|---|---|---|
| O/W formula | | % | % | % | % | % | % |
| | INCI-Name | w/w | w/w | w/w | w/w | w/w | w/w |
| Part A | Butyl Methoxydibenzoylmethane | 4.00 | 4.00 | 4.00 | 4.00 | 4.00 | 4.00 |
| | Polysorbate 80 | 1.13 | 1.13 | 1.13 | 1.13 | 1.13 | 1.13 |
| | Dibutyl Adipate | 20.00 | 20.00 | 20.00 | 20.00 | 20.00 | 20.00 |
| Part B | Aqua | Qs. to 100 | Qs. to 100 | Qs. to 100 | Qs. to 100 | Qs. to 100 | Qs. to 100 |
| | Xanthan Gum | 0.20 | 0.20 | 0.20 | 0.20 | 0.20 | 0.20 |
| Part C | Sodium Acrylates Copolymer (and) | 3.75 | 3.75 | 3.75 | 3.75 | 3.75 | 3.75 |
| | Mineral Oil (and) PPG-1 Trideceth-6 | | | | | | |
| Part D | Propylene Glycol (and) Diazolidinyl | 0.80 | 0.80 | 0.80 | 0.80 | 0.80 | 0.80 |
| | Urea (and) Methylparaben (and) Propylparaben | | | | | | |
| Part E | Triethanolamine | 0.00 | 4.00 | 2.00 | 0.00 | 0.00 | 0.00 |
| | Ethanolamine | 0.00 | 0.00 | 0.00 | 4.00 | 0.00 | 0.00 |
| | Diethanolamine | 0.00 | 0.00 | 0.00 | 0.00 | 0.00 | 4.00 |
| | Aminomethyl Propanol | 0.00 | 0.00 | 0.00 | 0.00 | 4.00 | 0.00 |
| Part F | Dispersion 1 or dispersion 2 as described | 8.00 | 8.00 | 8.00 | 8.00 | 8.00 | 8.00 |
| | in Table 1 | | | | | | |

### Manufacturing Instruction:

Heat part A and B separately to 75° C. Add part A to part B under continuous stirring. Add part C immediately. Let cool down to 50° C. and add part D, add part E under stirring during 5 minutes and add part F at room temperature.

### Characterization Methods:

Storage at RT and 50° C. during 3 weeks. Microscopy (under polarized light) with Leitz Diaplan microscope, MPF curves from 290 nm to 400 nm with Labsphere UV Transmittance analyzer and Sensory testing.

TABLE 1
Composition of micronized amino substituted hydroxyphenyl benzophenone derivatives

| Dispersion | | |
|---|---|---|
| 1 | Ingredients | Dispersion 2 |
| 50% | 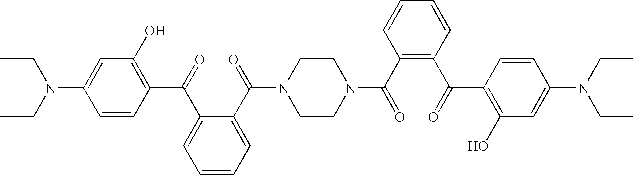 | 45-50% |
| 10% | aqueous formulation of 51-55% Decyl Glucoside | — |
| 1.5% | Potassium Cetyl Phosphate | — |
| | aqueous formulation of 26-28% Sodium Myreth Sulfate | 18.5% |
| 0.1% | Xanthan Gum | 0.1% |
| 0.2% | 1,3 Butanediol | 0.2% |
| 0.5-1% | anti-foam emulsion containing 20% Simethicone. | 0.5-1% |
| ad 100% | Water deion. | ad 100% |
| 120-140 | Particle size (d50) in nm | 120-140 |

### Results:

### Microscopy

### Examples 2, 3, 4 and 6

Crystal needles (1-200 μm) may be observed under polarized light within the formulations.

Speed and intensity of the formations of large particles are summarized in the Table 2.

### Example 5

No particle growth observed after 3 weeks at 50° C.

TABLE 2
Microscopy investigation of formulas of Examples 1 to 6

| O/W formulation | | Ex. 1 | Ex. 2 | Ex. 3 | Ex. 4 | Ex. 5 | Ex. 6 |
|---|---|---|---|---|---|---|---|
| RT | 1 day | − | − | − | − | − | − |
| | 3 weeks | ++ | + | ++ | + | − | + |
| 50° C. | 1 day | ++ | + | + | − | − | + |
| | 3 weeks | +++ | ++ | +++ | ++ | − | ++ |

+++: strong (many crystal needles)
++: crystal needles
+: few crystal needles
−: no particle growth observed

### Sensory Aspect

After 3 weeks at 50° C.

| | |
|---|---|
| Examples 4 and 5: | shiny and fluid |
| Examples 2, 3 and 6: | less shiny and less fluid |
| Example 1: | mat, grainy and viscous |

1. A cosmetic composition comprising
(a) 0.1 to 20% b.w. of a micronized benzophenone UV absorber of formula (1),
(b) 0.1 to 10% b.w. of a methoxydibenzoylmethane derivative and
(c) 0.1 to 50% b.w. of an amine or an amide, 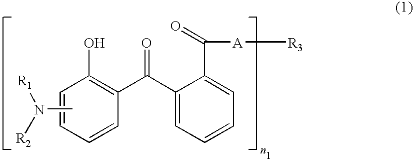
wherein
R₁ and R₂ independently from each other are C₁-C₂₀alkyl; C₂-C₂₀alkenyl; C₃-C₁₀cycloalkyl; C₃-C₁₀cycloalkenyl; or R₁ and R₂ together with the linking nitrogen atom form a 5- or 6-membered heterocyclic ring;
n₁ is a number from 1 to 4;
when n₁=1,
R₃ is a saturated or unsaturated heterocyclic radical; hydroxy-C₁-C₅alkyl; cyclohexyl optionally substituted with one or more C₁-C₅alkyl; or phenyl optionally substituted with a heterocyclic radical, aminocarbonyl or C₁-C₅alkylcarboxy;
when n₁ is 2,
R₃ is an alkylene-, cycloalkylene, alkenylene or phenylene radical which is optionally interrupted by —O—, —NH—, a carbonyl- or a carboxy group; a radical of formula *—CH₂—C≡C—CH₂—*; or R₃ together with A forms a bivalent radical of the formula 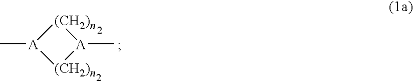
wherein
n₂ is a number from 1 to 3;
when n₁ is 3,
R₃ is an alkantriyl radical;
when n₁ is 4,
R₃ is an alkanetetrayl radical;
A is —O—; or —N(R₅)—; and
R₅ is hydrogen; C₁-C₅alkyl; or hydroxy-C₁-C₅alkyl.

2. (canceled)

3. A composition according to claim 1, where in formula (1)
R₁ and R₂ independently from each other are hydrogen; C₁-C₂₀alkyl; C₂-C₂₀alkenyl; C₃-C₁₀cycloalkyl; C₃-C₁₀cycloalkenyl; or R₁ and R₂ together with the linking nitrogen atom form a 5- or 6-membered heterocyclic ring;
n₁ is a number from 1 to 4;
when n₁ is 1,
R₃ is a saturated or unsaturated heterocyclic radical; hydroxy-C₁-C₅alkyl; or Cyclohexyl substituted with one or more C₁-C₅alkyl;
when n₁ is 2,
R₃ is an alkylene-, cycloalkylene- or alkenylene radical which is optionally interrupted by a carbonyl- or carboxy group;
when n₁ is 3,
R₃ is an alkantriyl radical;
when n₁ is 4,
R₃ is an alkanetetrayl radical;
A is —O—; or —N(R₅)—; and
R₅ is hydrogen; C₁-C₅alkyl; or hydroxy-C₁-C₅alkyl.

4. A composition according to claim 1, where in formula (1)
R₁ and R₂ are C₁-C₂₀alkyl.

5. A composition according to claim 1, where in formula (1)
R₁ and R₂ have the same definition.

6. A composition according to claim 1, where in formula (1)
n₁ is 2 and
R₃ is a C₁-C₁₂alkylene radical.

7. A composition according to claim 1, where in formula (1)
n₁ is 2;
R₃ is a radical of formula 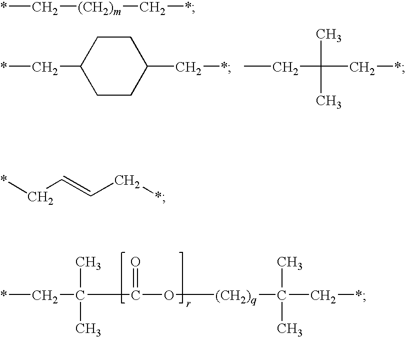
or R₃ together with A forms a bivalent radical of the formula 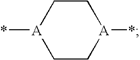
r is 0; or 1; and
q is a number from 0 to 5.

8. A composition according to claim 1, wherein the benzophenone UV absorber corresponds to formula (3) 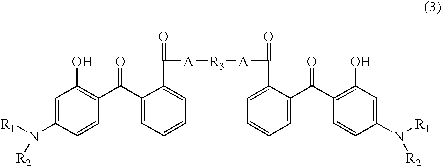
wherein
R₁ and R₂ independently from each other are hydrogen; or C₁-C₅alkyl;
A is —NH; or —O—; and
R₃ is a C₁-C₁₂alkylene radical; or R₃ together with A forms a bivalent radical of the formula

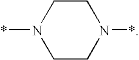

9. A composition according to claim 1, wherein the methoxydibenzoylmethane is 4-(tert-butyl)-4′-methoxydibenzoylmethane.

10. A composition according to claim 1, comprising amines selected from mono-, di-, and triethanolamines.

11. A composition according to claim 1, comprising amines selected from aminomethyl Propanol (AMP), ethanolamine (EA), diethyl ethanolamine (DEA), tromethamine (Tris Amino) and triethanolamine (TEA).

12. A composition according to claim 1, comprising amines selected from amine oxides of formula (6) RR′R″NO, wherein
R is C₁₂-C₃₀alkyl; and
R′ and R″ independently from each other are C₁-C₁₂alkyl.

13. A composition according to claim 1, comprising amines selected from alkoxylated amines of formula (4a) or (4b) 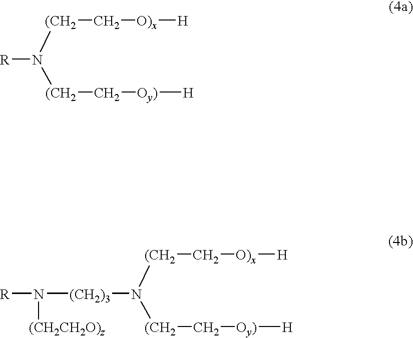
wherein
R is C₁-C₃₀alkyl; and
X, y and z independently from each other are a number from 1 to 20.

14. A composition according to claim 1, comprising amines selected from alkylamido alkylamines of formula (5) 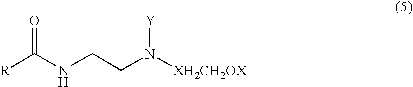
wherein
R is C₁-C₃₀alkyl;
X is hydrogen; or —CH₂COO—Na+ and
Y is —CH₂COO—Na+; —CH₂CH₂COO—Na+, —CH₂CHOHCH₂SO₃—Na+, or —CH₂CHOHCH₂OPO₃⁻H+Na+.

15. A composition according to claim 1, comprising amides selected from alkanolamides of formula (2) 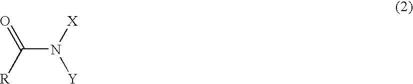
wherein
R is C₁-C₃₀alkyl;
X is —CHR′CH₂OH;
R′ is hydrogen; or C₁-C₁₂alkyl; and
Y is —CHR′CH₂OH; C₁-C₁₂alkyl; or C₁-C₁₂hydroxyalkyl.

16. A composition according to claim 1, comprising amides selected from alkoxylated amides of formula (3a) or (3b) 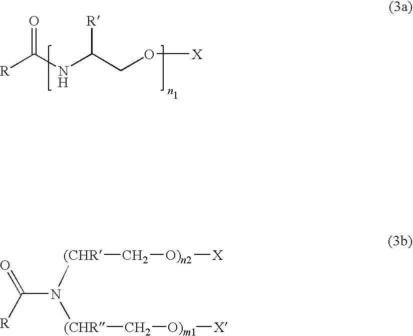
wherein
R is C₁-C₃₀alkyl;
R′ and R″ independently from each other are hydrogen; or C₁-C₁₂alkyl;
X and X′ independently from each other are hydrogen; or sulfosuccinyl;
n₁ is a number from 2 to 60;
n₂ and m₁ independently from each other are a number from 1 to 60, and the sum of n₂+m₁≧3.

17. A composition according to claim 1, comprising amines selected from mono-, di-, and triethanolamines, and where
the micronized UV absorber corresponds to formula (3) 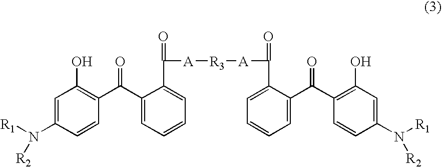
wherein
R₁ and R₂ independently from each other are hydrogen; or C₁-C₅alkyl;
A is —NH; or —O—; and
R₃ is a C₁-C₁₂alkylene radical; or R₃ together with A forms a bivalent radical of the formula) 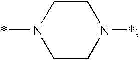
and the methoxydibenzoylmethane derivative is 4-(tert-butyl)-4′-methoxydibenzoylmethane.

18-21. (canceled)
